(19) United States
(12) Patent Application Publication
(10) Pub. No.: US 2006/0025800 A1
(43) Pub. Date: Feb. 2, 2006

(54) Method and device for surgical ventricular repair

(76) Inventor: Mitta Suresh, Richardson, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/158,293

(22) Filed: Jun. 21, 2005

Related U.S. Application Data
(63) Continuation-in-part of application No. 10/790,669, filed on Mar. 1, 2004.
Application No. 10/790,669 is a continuation-in-part of application No. 10/235,295, filed on Sep. 5, 2002.
(60) Provisional application No. 60/317,197, filed on Sep. 5, 2001.
(60) Provisional application No. 60/327,221, filed on Oct. 5, 2001.

(51) Int. Cl. A61M 29/00

(52) U.S. Cl. 606/198

(57) ABSTRACT

Embodiments disclose a method for repairing a heart of a human. A method may include introducing a collapsed reinforcing element through the skin into the vascular system of the human. The method may include delivering the reinforcing element into a left ventricle through the arteries. Once inside the left ventricle, the reinforcing element may be expanded to an expanded shape. In certain embodiments, a reinforcing element may be used to structurally reinforce a portion of an endocardial surface of a heart. The reinforcing element may include a preshaped patch and/or a plurality of preshaped flexible conduits. The method may include deploying the reinforcing element soon after a myocardial infarction to inhibit naturally occurring remodeling of the heart. The reinforcing element may be deployed with or without the use of a shaper. In some embodiments, a reinforcement element may be positioned on/coupled to an external surface of a human heart. In some embodiments, a reinforcing element may include an externally positioned apparatus configured to substantially reshape a portion of an interior chamber of a heart.

### PRIORITY CLAIM

This application is a continuation-in-part of U.S. patent application Ser. No. 10/790,669 entitled “METHOD AND DEVICE FOR PERCUTANEOUS SURGICAL VENTRICULAR REPAIR” filed on Mar. 1, 2004, which is a continuation-in-part of U.S. patent application Ser. No. 10/235,295 entitled “METHOD AND DEVICE FOR PERCUTANEOUS SURGICAL VENTRICULAR REPAIR” filed on Sep. 5, 2002, which claims priority to U.S. Provisional Patent Application Ser. No. 60/317,197 entitled “DEVICE AND METHOD FOR ENDOSCOPIC SURGICAL VENTRICULAR REPAIR” filed on Sep. 5, 2001, and U.S. Provisional Patent Application Ser. No. 60/327,221 entitled “METHOD AND DEVICE FOR CLOSED CHEST PLACEMENT OF SEPTUM” filed on Oct. 5, 2001, the disclosures of which are hereby incorporated by reference.

### BACKGROUND OF THE INVENTION

1. Field of the Invention

This invention relates generally to surgical methods and apparatuses for performing surgical ventricular repair endoscopically and/or through a minimally invasive incision.

2. Description of the Related Art

The function of a heart in an animal is primarily to deliver life-supporting oxygenated blood to tissue throughout the body. This function is accomplished in four stages, each relating to a particular chamber of the heart. Initially deoxygenated blood is received in the right auricle of the heart. This deoxygenated blood is pumped by the right ventricle of the heart to the lungs where the blood is oxygenated. The oxygenated blood is initially received in the left auricle of the heart and ultimately pumped by the left ventricle of the heart throughout the body. It may be seen that the left ventricular chamber of the heart is of particular importance in this process as it is relied upon to pump the oxygenated blood initially through a mitral valve into and ultimately throughout the entire vascular system.

The shape and volume of the normal heart are of particular interest as they combine to dramatically affect the way that the blood is pumped. The left ventricle which is the primary pumping chamber, is somewhat elliptical, conical, or apical in shape in that it is longer (the longest portion of the long axis of the left ventricle extending roughly from the aortic valve to the apex) than it is wide (short axis the widest portion of the short axis of the left ventricle extending roughly from the ventricle wall to the septum). The left ventricle descends from a base with a decreasing cross-sectional circumference, to a point or apex. The left ventricle is further defined by a lateral ventricle wall and a septum, which extends between the auricles and the ventricles.

Two types of motion accomplish the pumping of the blood from the left ventricle. One of these motions is a simple squeezing motion, which occurs between the lateral wall and the septum. The squeezing motion occurs as a result of a thickening of the muscle fibers in the myocardium. This compresses the blood in the ventricle chamber and ejects it into the body. The thickening changes between diastole and systole. This is seen easily by echocardiogram, PET, and MRI imaging and may be routinely measured.

The other type of motion is a twisting or writhing motion, which begins at the apex and rises toward the base. The rising writhing motion occurs because the heart muscle fibers run in a circular or spiral direction around the heart. When these fibers constrict they cause the heart to twist initially at the small area of the apex, but progressively and ultimately to the wide area of the base. These squeezing and twisting motions are equally important, as they are each responsible for moving approximately one-half of the blood pumped. The contractility or stiffness of these fibers are major determinants in how well the ventricle pumps.

The amount of blood pumped from the left ventricle divided by the amount of blood available to be pumped is referred to as the ejection fraction of the heart. Generally, a healthier heart has a higher ejection fraction. A normal heart, for example may have a total volume of one hundred milliliters and an ejection fraction of sixty percent. Under these circumstances, 60 milliliters of blood are pumped with each beat of the heart. It is this volume in the normal heart of this example that is pumped with each beat to provide nutrients including oxygen to the muscles and other tissues of the body.

Realizing that the heart is part of the body tissue, and the heart muscle also requires oxygenated blood, it may be appreciated that the normal function of the heart is greatly upset by clotting or closure of the coronary arteries. When the coronary arteries are blocked, an associate portion of the heart muscle becomes oxygen-starved and begins to die. This is clinically referred to as a heart attack. Ischemic cardiomyopathy typically occurs as the rest of the heart dilates in an attempt to maintain the heart's output to the body.

As the ischemia progresses through its various stages, the affected myocardium dies and loses its ability to contribute to the pumping action of the heart. The ischemic muscle is no longer capable of contracting so it may not contribute to either squeezing or twisting motion required to pump blood. This non-contracting tissue is said to be akinetic. In severe cases the akinetic tissue, which is not capable of contracting, is in fact elastic so that blood pressure tends to develop a bulge or expansion of the chamber. This muscle tissue is not only akinetic, in that it does not contribute to the pumping function, but it is in fact dyskinetic, in that it detracts from the pumping function. This is particularly detrimental to the limited pumping action available, as the heart loses even more of its energy to pumping the bulge instead of the blood.

The body seems to realize that with a reduced pumping capacity, the ejection fraction of the heart is automatically reduced. For example, the ejection fraction may drop from a normal sixty percent to perhaps twenty-percent. Realizing that the body still requires the same volume of blood for oxygen and nutrition, the body causes its heart to dilate or enlarge in size so that the smaller ejection fraction pumps about the same amount of blood. As noted, a normal heart with a blood capacity of seventy milliliters and an ejection fraction of sixty percent would pump approximately 42 milliliters per beat. The body seems to appreciate that this same volume per beat may be maintained by an ejection fraction of only thirty-percent if the ventricle enlarges to a capacity of 140 milliliters. This increase in volume, commonly referred to as “remodeling,” not only changes the volume of the left ventricle, but also its shape. The heart becomes greatly enlarged and the left ventricle becomes more spherical in shape losing its apex.

On the level of the muscle fibers, it has been noted that dilation of the heart causes the fibers to reorient themselves so that they are directed away from the inner heart chamber containing the blood. As a consequence, the fibers are poorly oriented to accomplish even the squeezing action, as the lines of force become less perpendicular to the heart wall. This change in fiber orientation occurs as the heart dilates and moves from its normal elliptical shape to its dilated spherical shape. The spherical shape further reduces pumping efficiency since the fibers which normally encircle the apex facilitate writhing are changed to a more flattened formation as a result of these spherical configurations.

Of course, this change in architecture has a dramatic effect on wall thickness, radius, and stress on the heart wall. In particular, it will be noted that absent the normal conical shape, the twisting motion at the apex, which may account for as much as one half of the pumping action, is lost. As a consequence, the more spherical architecture must rely almost totally on the lateral squeezing action to pump blood. This lateral squeezing action is inefficient and very different from the more efficient twisting action of the heart.

Although the dilated heart may be capable of sustaining life, it is significantly stressed and rapidly approaches a stage where it may no longer pump blood effectively. In this stage, commonly referred to as congestive heart failure, the heart becomes distended and is generally incapable of pumping blood returning from the lungs. This further results in lung congestion and fatigue. Congestive heart failure is a major cause of death and disability in the United States where approximately 400,000 cases occur annually.

What is needed therefore is a reliable method and apparatus to allow a surgeon to perform surgical ventricular repair, preferably without having to do a full sternotomy and/or make large incisions in the chest. Additionally, such methods could be performed on a beating heart eliminating the need for lengthy full bypass circuit runs.

### SUMMARY

In response to these and other problems, an improved apparatus and method is provided for endoscopic surgical ventricle repair which allows a surgeon to perform a surgical ventricular repair procedure through a closed chest or through a small thoracotomy on a beating, fibrillating, or an arrested heart. In some embodiments, there is a method for repairing a heart of a human. The method may include introducing a shaping device percutaneously into a vasculature of a human. The shaping device may be in a collapsed state during delivery. The method may include delivering the shaping device into a left ventricle through the vasculature. The method may include expanding the shaping device to an expanded shape after entering the left ventricle. In certain embodiments, the method may include imbricating a wall of the ventricle over the shaping device. The method may include collapsing the shaping device, and removing the shaping device from the left ventricle such that the ventricle is restored to an appropriate size.

In response to the problems mentioned above, there is disclosed an embodiment of a reinforcing element to be used in ventricular repair. “Reinforcing” in the context of this application is to strengthen or make stronger by patching, propping, adding new material, etc. For example, in certain embodiments of this application a reinforcing element is attached to the endocardial surface of a human heart ventricle, thereby strengthening the wall by adding supportive material to the wall. In an embodiment, a reinforcing element may be used to repair a ventricle (e.g., a left ventricle) of a human heart. The reinforcing element may have a first and a second predetermined shape. The reinforcing element has a second predetermined shape that is substantially the same as the shape of a portion of the ventricle. In an embodiment, a reinforcing element may have a second predetermined shape including a conical shape with a round apex. When inserted into a patient, the tip of the cone may act as an apex of the heart. The reinforcing element may releasably attach to an endocardial surface of the heart. The reinforcing element may assist in reforming or reconstructing a ventricle. The reinforcing element may assist in reforming a contour of a ventricle. In some embodiments, a reinforcing element may inhibit expansion of an endocardial surface. The area of the endocardial surface may be averaged over a specified time period (e.g., a cardiac cycle) or phase. The reinforcing element may inhibit expansion of an endocardial surface while allowing normal contraction and expansion of the heart. The reinforcing element may function to inhibit expansion of the interior wall of the left ventricle (e.g., endocardial surface). The reinforcing element may function to inhibit expansion of the volume of the ventricle. In some embodiments, a reinforcing element may inhibit expansion of the end diastolic volume of the left or right ventricle. “End diastolic volume” in the context of this application is generally defined as the amount of blood in the ventricle immediately before a cardiac contraction begins; a measurement of cardiac filling between beats, related to diastolic function. In some embodiments, a reinforcing element may inhibit expansion of the diastolic size of the interior/endocardial wall of the left or right ventricle. “End diastolic size” in the context of this application is generally defined as the surface area of the interior/endocardial surface of the wall of the ventricle immediately before a cardiac contraction begins.

In some embodiments, a reinforcing element may be made from a bio-prosthetic like a porcine or bovine pericardium, or a prosthetic material like polyester or PTFE. Such a product may be fabricated by weaving or knitting or by using one or more continuous sheets. Optionally, the reinforcing element could have radiopaque markings. Furthermore, the reinforcing element could be made of ion exchange material, which can act as an artificial muscle.

An “endocardial surface” is generally defined as an interior surface of a wall of a portion of a human heart (e.g., a left or right ventricle).

In some embodiments, a reinforcing element may include a plurality of conduits forming a predetermined shape. The reinforcing element may be collapsible. A collapsible reinforcing element may allow a user to insert the reinforcing element percutaneously. The reinforcing element may include a coupling mechanism controlled by an activation mechanism. The coupling mechanism may function to releasably couple/attach the reinforcing element to a portion of an interior wall (e.g., endocardial) surface of a heart. Couple or attach is to be generally defined as being directly attached (touching) or indirectly attached (something between the reinforcement element and the wall).

In some embodiments, a reinforcing element may be attached to an exterior wall surface of a portion of a heart (e.g., a left ventricle). The reinforcing element may include a three-dimensional shape. The reinforcing element may include a first side and a second side. The second side may be positioned substantially adjacent the portion of the heart. The interior portion of the heart may substantially mimic a contour of the second side, effectively reshaping at least one interior chamber of the heart.

The procedure addresses the ability of the surgeon to perform a surgical ventricular repair procedure that allows the surgeon to ensure that he gets the intended size and shape of the ventricle with an apex while at the same time excluding all the akinetic and dyskinetic tissue.

The procedure, by excluding much, if not all, of the akinetic and dyskinetic tissue while allowing the surgeon to create the proper shape with an apex, significantly reduces stress on the heart muscle and improves surgical outcome. The procedure, by being done with a precise device allows the surgeon to make the procedure repeatable and reliable. The device takes the variation out of the procedure.

In some embodiments, a system and/or method may be directed to supporting and or to reinforcing myocardial tissue in the region of an infarct site. The system and/or method may cover at least a section of an infarct site. The system may allow a heart to pump more efficiently. The system may inhibit continued remodeling of the akinetic and/or dyskinetic heart tissue. The system may inhibit dilated cardiomyopathy. The system may inhibit aneurismal tissue from forming. The system may inhibit any of the various complications these (and other) conditions may cause. The device may include a feature to stabilize, strengthen, bind, cross link, arrest, and/or delay the tissue remodeling process, or otherwise directly reinforce the infarcted myocardial tissue itself.

In some embodiments, a system may include a patch system. The patch system may include a mechanism for coupling the patch to human tissue (e.g., cardiac tissue). In some embodiments, a patch system may include therapeutic functions. Therapeutic functions may include systems (e.g., pharmaceutical agents) which assist in regenerating human tissue and function. A patch system may include direct therapeutic infusion systems. Therapeutic agents may be directed directly into and around infarcted myocardial tissue. Systems and/or methods may include thermal tissue treatment into and/or around infarcted myocardial tissue.

In some embodiments, a system may include a reinforcing element for reshaping at least a portion of a human heart. The reinforcing element may function to attach to a portion of the epicardial surface of a human heart. The reinforcing element may function to attach to a portion of the epicardial surface of a left or right ventricle of the heart. A reinforcing element may function to change a dimension of at least a portion of a human heart. In some embodiments, a reinforcing element may function to change a size and/or shape of an interior space in a left or right ventricle.

In some embodiments, a reinforcing element may attach to a portion of the epicardial surface of a left or right ventricle of the heart. The reinforcing element may change from a first shape to a second shape upon activation and thereby change a dimension of at least a portion of the ventricle.

In some embodiments, an epicardial surface of a human heart may include at least some scar tissue. In some embodiments, an epicardial surface of a human heart may include at least some infarcted tissue.

The reinforcing element may function to inhibit expansion of an average of an endocardial surface over a cardiac cycle.

In some embodiments, the reinforcing element may include one or more portions which facilitate post operative evaluation of a positioned reinforcing element. At least some of the portions of the reinforcing element which facilitate post operative evaluation may be MRI scan sensitive.

At least some portion of a reinforcing element may include one or more layers coupled together.

A reinforcing element may be coupled or attached to an epicardial surface of a heart in a variety of ways known to one skilled in the art. In some embodiments, a reinforcing element may be attached to an epicardial surface using sutures. The reinforcing element may be attached to a portion of the epicardial surface using staples. The reinforcing element may be attached to a portion of the epicardial surface using biocompatible adhesives. In some embodiments, a reinforcing element may function to releasably attach to a portion of the epicardial surface of the heart

In some embodiments, a reinforcing element may function to inhibit expansion of an average of an endocardial surface over a cardiac cycle of the left or right ventricle.

In some embodiments, a reinforcing element may function to inhibit expansion of an endocardial surface such that substantially normal contraction and expansion during a cardiac cycle of the heart remains substantially unimpeded. Normal contraction and expansion of a heart may be generally defined as contraction and expansion within desired parameters of a healthy heart as defined by a specific patients specifications (e.g., gender, age, height, weight).

In some embodiments, a reinforcing element may include an adjustment mechanism. The adjustment mechanism may function, upon activation, to change a dimension of at least a portion of the reinforcing element. In some embodiments, an adjustment mechanism may include an inflatable portion which functions, upon activation, to change a dimension of at least a portion of the reinforcing element. The inflatable portion may be positioned between an outer surface of the adjustment mechanism and the epicardial surface of the left or right ventricle. In some embodiments, a system may include an inflation mechanism. The inflation mechanism may be removably coupled to the inflatable portion of the reinforcing element. The inflation mechanism may function to convey fluids to the inflatable portion. Fluids may include gasses (e.g., air, inert gasses (e.g., nitrogen, argon)) and/or liquids (e.g., water, gels).

In some embodiments, a reinforcing element may include an adjustment mechanism (as described herein) and an engagement mechanism. The engagement mechanism may function to inhibit an activated adjustment mechanism from moving. The engagement mechanism may function to inhibit an activated adjustment mechanism from moving such that

In some embodiments, a reinforcing element may include an activation mechanism. The activation mechanism may function to attach the reinforcing element to a portion of the surface of the heart.

In some embodiments, a reinforcing element may include a patch. The patch may include two or more openings. The openings may allow elongated members to pass through the openings. Upon activation the elongated members may function to change a dimension of at least a portion of the reinforcing element, and to thereby change a dimension of at least a portion of the ventricle. In some embodiments, the elongated members may include staples. In some embodiments, elongated members may include sutures.

In some embodiments, a reinforcing element may include a patch. The patch may be formed from a system of elongated members. Upon activation the elongated members may change a dimension of at least a portion of the reinforcing element, and to thereby change a dimension of at least a portion of the ventricle. In some embodiments, the elongated members may be coupled to an adjustment mechanism. Upon activation of the adjustment mechanism the elongated members may change a dimension of at least a portion of the ventricle.

In some embodiments, a reinforcing element may be formed from shape memory materials (e.g., nitinol).

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 depicts an embodiment of a method of repairing at least a portion of a human heart.

FIG. 2a depicts an embodiment of a shaping device.

FIG. 2b depicts an embodiment of a shaping device in an expanded condition.

FIG. 2c depicts an embodiment of a shaping device in a collapsed condition.

FIG. 2d depicts an embodiment of a shaping device in an expanded condition.

FIG. 2e depicts an embodiment of a shaping device in a collapsed condition.

FIG. 3a depicts an embodiment of a shaping device deployed within a human heart.

FIG. 3b depicts an embodiment of a human heart before remodeling.

FIG. 4 depicts an embodiment of a shaping device deployed within a human heart.

FIG. 5 depicts an embodiment of a method of repairing at least a portion of a human heart.

FIG. 6 depicts an embodiment of a method of repairing at least a portion of a human heart.

FIG. 7 depicts an embodiment deployed within a human heart.

FIG. 8a depicts an embodiment deployed within a human heart.

FIG. 8b depicts an embodiment deployed within a human heart.

FIG. 8c depicts an embodiment deployed within a human heart.

FIG. 8d depicts an embodiment deployed within a human heart.

FIG. 9 depicts an embodiment of a newly infarcted left ventricle with anterior-apical scar.

FIG. 10 depicts an embodiment of the ventricle in depicted in FIG. 9.

FIG. 11 depicts an embodiment of the ventricle in depicted in FIG. 9 including a reinforcing element.

FIG. 12 depicts an embodiment of a ventricle including a reinforcing element after a period of time has passed since placement of the reinforcing element.

FIG. 13 depicts an embodiment of a reinforcing element.

FIG. 14 depicts an embodiment of a shaper that matches the object in FIG. 13.

FIG. 15 depicts an embodiment of a reinforcing element with a coupling mechanism in an activated/engaged state.

FIG. 16 depicts an embodiment of a portion of a reinforcing element including a sectional view of one conduit of the reinforcing element with a coupling mechanism in an inactivated/disengaged state.

FIG. 17 depicts an embodiment of a portion of a reinforcing element including a sectional view of the reinforcing element with coupling mechanism in an activated/engaged position.

FIG. 18 depicts an embodiment of a portion of a reinforcing element with a coupling mechanism in an inactivated/disengaged state.

FIG. 19 depicts an embodiment of a portion of a reinforcing element with a coupling mechanism in an activated/engaged state.

FIG. 20 depicts an embodiment of a portion of a reinforcing element with a coupling mechanism in an activated/engaged state positioned in a left ventricle of a heart wherein the portions of the coupling mechanism extend partially into an endocardial wall surface.

FIG. 21 depicts an embodiment of a portion of a reinforcing element with a coupling mechanism in an activated/engaged state positioned in a left ventricle of a heart wherein the portions of the coupling mechanism extend through an endocardial wall surface.

FIG. 22 depicts an embodiment of a portion of a reinforcing element with a coupling mechanism.

FIG. 23 depicts an embodiment of a portion of a reinforcing element with a coupling mechanism positioned in a left ventricle of a heart wherein the portions of the coupling mechanism extend through an endocardial wall surface.

FIG. 24 depicts an embodiment of a reinforcing element.

FIG. 25 depicts an embodiment of a reinforcing element cut to be placed in a patient.

FIG. 26 depicts an embodiment of a sectional view of a dilated heart with a reinforcing element.

FIG. 27 depicts an embodiment of a reinforcing element including an adjustment mechanism in an inactivated state.

FIG. 28 depicts an embodiment of a reinforcing element including an adjustment mechanism in an activated state.

FIG. 29 depicts an embodiment of a portion of a reinforcing element including a sectional view of the reinforcing element with adjustment mechanism in an inactivated/disengaged position.

FIG. 30 depicts an embodiment of a portion of a reinforcing element including a sectional view of the reinforcing element with adjustment mechanism in an activated/engaged position.

FIG. 31 depicts an embodiment of a reinforcing element.

FIG. 32 depicts an embodiment of a reinforcing element coupled to a portion of a human heart during use.

FIG. 33 depicts an embodiment of a method for positioning a reinforcing element.

FIG. 34 depicts an embodiment of a human heart including a depiction of infarcted tissue.

FIG. 35 depicts an embodiment of a human heart with an area of infarcted tissue.

FIG. 36 depicts an embodiment of a human heart with suture or staples coupled to the heart over the infarcted tissue.

FIG. 37 depicts an embodiment of a human heart with suture or staples coupled to the heart over the infarcted tissue, before and after the infarcted area has been reduced.

FIG. 38 depicts an embodiment of a human heart with suture or staples coupled to the heart over the infarcted tissue, before the infarcted area has been reduced.

FIG. 39 depicts an embodiment of a patch including openings.

FIG. 40 depicts an embodiment of a wire mesh patch.

FIG. 41 depicts an embodiment of a patch including coupling mechanisms.

FIG. 42 depicts an embodiment of a patch including an inflatable/expandable portion.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

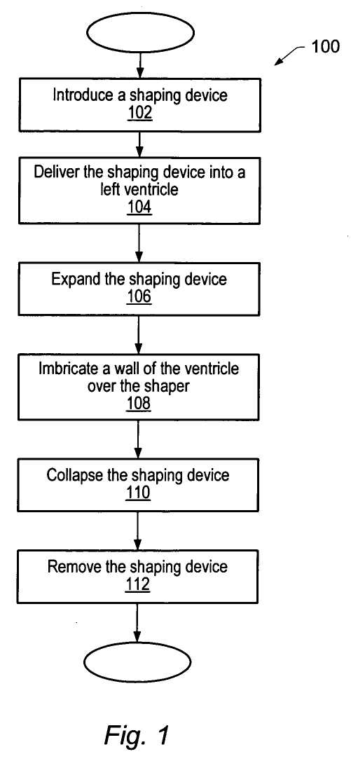

Turning to FIG. 1, there is presented an overview method 100 for performing and using an embodiment. Method 100 may use the following components: a shaping device, a patch, and/or a stapling device.

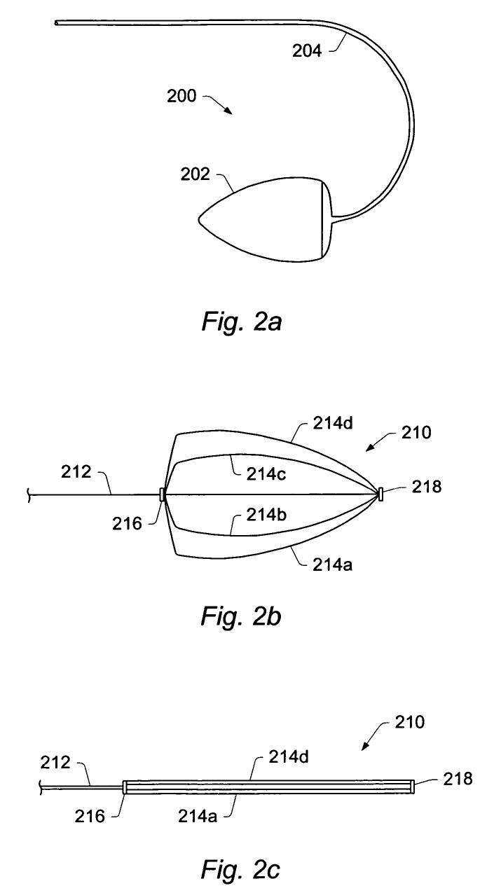

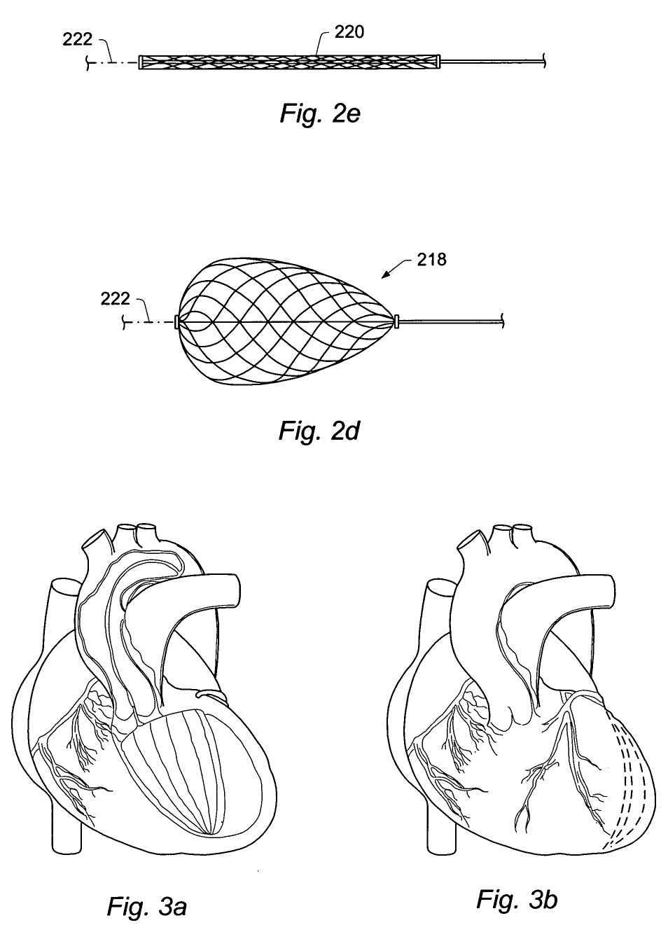

In some embodiments, a shaping device may be pre-shaped to generally model the appropriate volume and shape of the left ventricle, as is depicted in FIG. 2a. Shaping device 200 may be used as a guide in reforming the left ventricle so that the reconstructed heart may be formed closer to the size and shape of the pre-enlarged heart. Consequently, the heart performs better post operatively than with conventional methods. As illustrated in FIG. 2a, shaping device 200 may be conical or “tear drop” in shape. The length of shaping device 200 may vary with each patient and will typically be a function of the volume selected for the shaping device. The size, shape, and/or volume of shaping device 200 may vary according to individual patient specific needs. Shaping device 200 may be designed and manufactured for a specific patient's needs. In some embodiments, shaping device 200 may be manufactured in a variety of sizes, shapes, and/or volumes, from which a user may select an appropriate shaping device for a specific patient. Depending on the patient, the length may be between about three inches to about four inches to generally match the length of the pre-enlarged left ventricle. A doctor may select the appropriate volume for the shaping device by estimating the volume of the pre-enlarged left ventricle. Such selection procedures and shaping devices are discussed in U.S. patent application Ser. No. 09/864,510, filed on May 24, 2001 by the inventors, which is hereby incorporated by reference into this application.

In some embodiments, such as illustrated in FIG. 2a, the shaping device may be inflatable balloon 202 coupled to filler tube 204. Such tubes are well known in the art, and illustratively may be made of plastic-type materials such as PVC. A proximal end of filler tube 204 may be connected to a fluid reservoir (not shown), which may be used to fill a pre-specified amount of fluid into balloon 202 through filler tube 204. A fluid reservoir may include, for example, a syringe. The injection of fluid through filler tube 204 inflates balloon 202 to an inflated condition.

In certain embodiments, a shaping device may include a wire skeleton or frame, as illustrated in FIG. 2b. The wire frame could be made from surgical grade stainless steel, titanium, tantalum, and/or nitinol. Nitinol is a commercially available nickel-titanium alloy material that has shape memory and is super elastic. Nitinol medical products are available from AMF of Reuilly, France, and Flexmedics Corp., of White Bear Lake, Minn.

Shaping device 210 illustrated in FIG. 2b is in an expanded condition. In this embodiment, main wire 212 may run through the center of shaping device 210. Coupled to the main wire may be a series of back ribs 214a through 214d. Back ribs 214a through 214d may be coupled to collar 216.

FIG. 2c shows shaping device 210 in a collapsed position. In a collapsed position, back ribs 214a-214d surround main wire 212. During use, once shaping device 210 is inserted into the left ventricle, a user may cause collar 216 to slide along main wire 212 towards distal end 218 of the wire. The force exerted on collar 216 will cause the ribs to buckle radially outward as illustrated in FIG. 2b to a predetermined shape.

Some embodiments may include a wire mesh system such as illustrated in FIG. 2d. Wire mesh shaper 218 may be formed of a tubular fabric made from a plurality of wire strands. The wire strands forming wire mesh shaper 218 may have a predetermined relative orientation between the strands. Those skilled in the art will appreciate that the pick and pitch of the braided wires may be varied depending upon the desired density of the fabric. The tubular fabric may have metal strands which define two sets of essentially parallel generally spiraling and overlapping strands, with the strands of one set having a “hand”, i.e. a direction of rotation, opposite that of the other set. This tubular fabric is known in the fabric industry as a tubular braid.

The pitch of the wire strands (i.e., the angle defined between the turns of the wire and the axis of the braid), the pick of the fabric (i.e., the number of turns per unit length), the number of wires employed in a tubular braid, the size or diameter of each wire in the braid, and/or the diameter of the braid are all examples of considerations important in determining a number of important properties of the device. For example, the greater the pick and pitch of the fabric, and hence the greater the density of the wire strands in the fabric, the stiffer the device will be. The greater the diameter of each wire of the braid, the stiffer the device will be.

The wire strands of the tubular metal fabric may be manufactured from so-called shape memory alloys. Such alloys tend to have a temperature induced phase change which will cause the material to have a preferred configuration which may be fixed by heating the material above a certain transition temperature to induce a change in the phase of the material. When the alloy is cooled back down, the alloy will “remember” the shape it was in during the heat treatment and will tend to assume that configuration unless constrained from so doing.

Without any limitation intended, suitable wire strand materials may be selected from a group including a cobalt-based low thenrmal expansion alloy referred to in the field as ELGELOY, nickel-based high temperature high-strength “superalloys” (including nitinol) commercially available from, for example, Haynes International under the trade name HASTELLOY, nickel-based heat treatable alloys sold under the name INCOLOY by International Nickel, and/or a number of different grades of stainless steel. One important factor in choosing a suitable material for the wire strands is that the wires retain a suitable amount of the deformation induced by a molding surface when subjected to a predetermined heat treatment.

When the tubular braid, for example, is in its preformed relaxed configuration 218 as illustrated in FIG. 2d, the wire strands forming the tubular braid will have a first predetermined relative orientation with respect to one another. As the tubular braid is compressed along its axis 222, the fabric will tend to flare out away from axis 222 conforming to the shape of the mold. When the fabric is so deformed the relative orientation of the wire strands of the metal fabric will change. After undergoing the shape memory process, the resulting medical device has a preset relaxed configuration 218 as illustrated in FIG. 2d and a collapsed or stretched configuration 220 as illustrated in FIG. 2e, which allows the device to be passed through a catheter or other similar delivery device.

In some embodiments, the shaping device may also have mechanisms by which the epicardium may be grabbed and conformed to the shape of the shaping device. As will be explained below, in such an embodiment, the clasping instrument may be placed along the outer surface of the ventricle at precise locations and closed to take a bite out of the ventricle, reshaping the ventricle around the shaping device.

A delivery device or catheter (not shown) may take any suitable shape. In some embodiments, a delivery device may include an elongated flexible metal shaft having a threaded distal end. The delivery device may be used to urge the wire mesh shaper 218 through the lumen of a catheter for deployment in a channel of a patient's body. When the device is deployed out the distal end of the catheter, the device may still be retained by the delivery device. Once wire mesh shaper 218 is properly positioned, the distal end of the catheter maybe pressed against the medical device and the metal shaft or guidewire may be rotated about its axis to unscrew the medical device from the threaded distal end of the shaft. The catheter and guidewire may or may not be withdrawn at this point.

As will be explained below, in some embodiments of a method, a patch maybe used in method 100. In an embodiment, the patch may be made from sheet material. The patch may be a variety of shapes, including circular, elliptical, or triangular in shape. In certain embodiments, a sheet material for a patch may be formed from a biocompatible synthetic material, for example, from polyester (e.g., Dacron (Hemoshield™) manufactured by the DuPont Corporation), or polytetrafluoroethylene (e.g., Gortex™). The sheet material may also be autologous pericardium, or some other fixed mammalium tissue such as bovine pericardium or porcine tissue. The biocompatible synthetic material patch may be collagen impregnated to assist in hemostasis, or it may be sprayed with a hemostatic sealant to achieve better and instantaneous hemostasis.

In some embodiments, on one side of a patch, there may be a means of adhering the patch to the endocardium or inside of the heart. The patch may have markings that enable the movement and position of the patch to be post-operatively observed and analyzed under imaging systems, such as for example Magnetic Resonance Imaging (“MRI”), x-ray machines, fluoroscopy and/or other external visualization methods for post-operative clinical evaluation. Such markings will allow identification of the patch and allow for analysis of the heart's contractility in future post-operative evaluations. The markings may be radiopaque. Such radiopaque markings are discussed in U.S. patent application Ser. No. 09/864,510, filed on May 24, 2001 by the inventors, which has been incorporated by reference into this application.

In some embodiments, the shaping device may be coupled to the patch and/or have a mechanism, that couples to and releases the patch.

In certain embodiments, an imaging system may be used preoperatively to take MRI, PET, and/or echocardiography imaging data of the ventricle. Imaging data may be used to determine what the appropriate areas of the ventricle to exclude are and/or to determine what the appropriate volume of the ventricle should be.

Turning back now to FIG. 1, method 100 will now be discussed. For purposes of illustration only and not by way of limitation method 100 will now be discussed as part of a bypass procedure. The procedure may begin by a user positioning an endoscopic camera into the patient to view the infarcted area of a patient's heart. Preoperatively, the user may determine the size of the shaping device by selecting a shaping device that matches the volume of the ventricle desired for the particular patient.

When the shaping device is in a collapsed state, in 102, the shaping device may be introduced into the vasculature or vascular system of the patient. From the vascular system, in 104, the shaping device may be guided into the left ventricle.

In a bypass procedure, the femoral vein and artery are cannulated to connect the patient to the cardiopulmonary bypass machine. After the bypass machine is running, the shaping device is manipulated to deploy from a collapsed state to an expanded shape. In some embodiments, markings on the controlling handle will provide feedback to the user on how the shaping device is positioned so that he knows where the patch is in relation to the ventricle. A positioning device on the shaping device will align with an anatomical landmark inside the ventricle (e.g., the aortic annulus) to provide another reference location for the shaping device. In 106, the shaping device may be deployed into an expanded condition, as shown in FIG. 3a.

In 108, the wall of the ventricle may be imbricated over the shaping device, as shown in FIG. 3b. The term “imbricating” as used in this application generally means to bring together two edges of the ventricle wall that have non-viable tissue between them and excluding this portion of the ventricle wall, which will basically reshape the ventricle. The shaping device may help determine which edges should be brought together. However, some non-viable tissue may be left in the ventricle in order to reshape the ventricle to the appropriate size and shape.

In order to imbricate or reform the ventricle wall over the shaping device, a molding instrument may be inserted into the chest through a small opening in an intercostal space to reach the epicardium. This molding instrument will allow the surgeon to press the ventricle wall against the shaping device to help reshape the ventricle, as shown in FIG. 3b. This molding instrument may be withdrawn. A clasping instrument may be inserted. The molding instrument and clasping instrument may be one device. This clasping instrument will take portions or “bites” out of the ventricle wall starting at the edges of the area of non-viable tissue that needs to be excluded to restore the ventricle to its correct shape, size, and/or contour. The bites may be made with suture type devices, stapling devices, and/or clip type devices, for example. The clasping instrument may be partially closed to allow the user to ensure that he is properly shaping the ventricle onto the shaping device. If the user determines that he has the clasping instrument placed properly, the device will allow for full closure. The implements placed by the clasping instrument when closed will have pulled the ventricle wall over the shaping device and will maintain the ventricle's shape. Turning back to FIG. 1, once the shaping is complete, in 1 10, the shaping device may be collapsed and removed from the ventricle (112). In some embodiments, intraoperative imaging may be used during this procedure to aid the surgeon's view of the mandrel and/or ventricle interface.

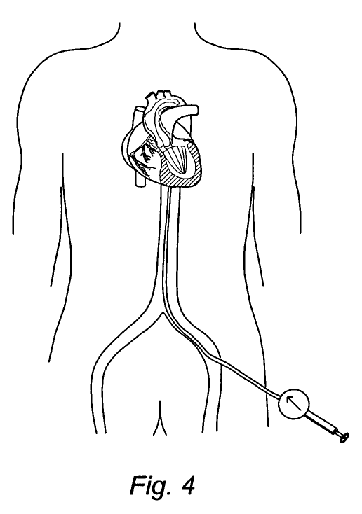

In certain embodiments, method 100 may be performed on a beating heart. Referring back to 102 of FIG. 1, the collapsed shaping device may be inserted into a femoral artery. In 104, the shaping device is passed through the femoral artery to the left ventricle as illustrated in FIG. 4. Once in the ventricle the shaping device may be expanded briefly and the user checks the alignment indicators to ensure that the patch is in the correct position. He collapses the mandrel and allows the heart to beat normally. This procedure minimizes the heart's contractions for a very brief period of time while the shaping device is deployed, but allows the heart to beat when the shaping device is collapsed. Once the surgeon has determined that the patient may tolerate another low flow period, the shaping device may be expanded again (106) and the wall of the ventricle is imbricated over the shaping device (108). The imbrication may be performed with the placement of clasping mechanisms. The placement of the clasping mechanisms may be determined from analysis of the preoperative imaging. A small opening may be made in an intercostal space and the clasping instrument inserted through this opening. Clasping mechanisms may now be placed on the ventricle and partially closed. The shaping device may be deployed again briefly and the user assesses the progress of the procedure and collapses the shaping device. If the clasping mechanisms are in the correct position, the shaping device is expanded again and the clasping mechanisms are closed fully. The deployed shaping device ensures that the ventricle is of the intended volume. In 110, the shaping device is collapsed and removed from the ventricle and the femoral artery (112).

In some embodiments, method 100 may be done as part of a thoracotomy, where the chest is opened in an intercostal space to allow greater access to the ventricle. A user could use the intercostal space opening to position the clasping instrument. If the user chooses, he could do revascularization of the lateral anterior descending artery along with the procedure. A cannula may be placed in the jugular vein to deliver cardioplegia to the coronary sinus, if the user desires to do the anastomosis on an arrested heart.

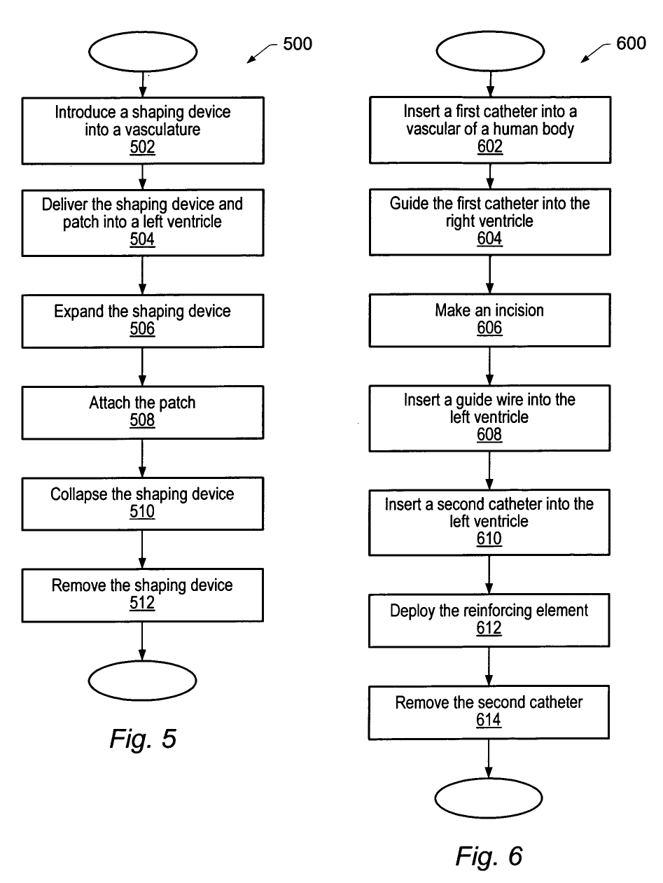

Turning to FIG. 5, method 500 will now be discussed. Method 500 may be used as either part of a bypass procedure or a beating heart procedure. As discussed in relation to method 100, a user may assess the location of the ventricle to be excluded (i.e., “the excluded portion”). The user may cut a patch to a size that covers the excluded portion or select a presized patch to match the excluded portion. The patch may be a predetermined size that corresponds to the size of the shaping device. The patch may be preattached to the shaping device. Once the size of the patch has been assessed, the user may select a shaping device that matches the volume of the ventricle desired for the particular patient. In some embodiments, the patch may be secured to the shaping device. The shaping device may be collapsed into/onto a delivery catheter.

When the shaping device is in a collapsed state, in 502 the shaping device and patch may be introduced into the vasculature or vascular system of the patient. From the vascular system, in 504, the shaping device and patch may be guided into the left ventricle.

In some embodiments, markings on the controlling handle may provide feedback to the user on how the shaping device is positioned, so that he knows where the patch is in relation to the ventricle. A positioning device on the shaping device may align with an anatomical landmark inside the ventricle (e.g., the aortic annulus) to provide another reference location for the shaping device. In 506, the shaping device may be deployed into an expanded condition, as illustrated in FIG. 7.

Once the molding instrument has been deployed, in 508, the patch may be attached to the epicardium of the heart. Once the shaping is complete, in 510, the shaping device will be collapsed and removed from the ventricle (512).

In some embodiments, the wall of the ventricle may be imbricated over the shaping device. In order to imbricate or reform the ventricle wall over the shaping device, a molding instrument may be inserted into the chest through a small opening in an intercostal space to reach the epicardium. This molding instrument may allow the user to press the ventricle wall against the shaping device. Pressing the ventricle wall against the shaping device may ensure that the patch gripping mechanism attaches to the ventricle wall that is to be excluded. This molding instrument may be withdrawn and a clasping instrument may be inserted. The molding instrument and clasping instrument may be one device. This clasping instrument may take portions or bites out of the ventricle wall starting at the edges of the area of non viable tissue that needs to be excluded to restore the ventricle to its correct shape, size and contour. The bites may be made with suture type devices and/or clip type devices, for example. The clasping instrument may be partially closed to allow the user to ensure that he is properly shaping the ventricle onto the shaping device. If the user determines that he has the clasping instrument placed properly, the device will allow for full closure. The implements placed by the clasping instrument when closed will have pulled the ventricle wall over the shaping device and will maintain the ventricle's shape. Intraoperative imaging may be used during this procedure to aid the user's view of the mandrel and ventricle interface.

Method 500 may be performed on a beating heart using intraoperative imaging. The shaping device, with the patch attached, may be passed through a femoral artery to the left ventricle. Once in the ventricle the shaping device may be expanded and an image made of the ventricle and the shaping device collapsed. This stops the heart for a very brief period of time while the shaping device is deployed, but allows the heart to beat when the shaping device is collapsed. Once the image is analyzed to ensure that the patch is in the proper place, the shaping device may be expanded again. The patch may be secured with the assistance of the molding instrument and the shaping device collapsed. The placement of the clasping mechanisms on the clasping instrument may be accomplished from analysis of the preoperative imaging. A small opening may be made in an intercostal space. The clasping instrument may be placed through this opening. Clasping mechanisms may now be placed on the ventricle and partially closed. The shaping device may be deployed again and another image taken of the ventricle and the shaping device collapsed. This image may be analyzed to ensure that the positioning of the clasping mechanisms is creating the desired shape of the ventricle over the shaping device. If the clasping mechanisms are in the correct position the shaping device may be expanded again and the clasping mechanisms are closed fully. The deployed shaping device ensures that the ventricle is the correct volume. The shaping device may then be collapsed and withdrawn from the ventricle and the femoral artery.

In some embodiments, a patch may be positioned in the ventricle separately from the shaping device. This procedure may be accomplished by having the patch introduced into the ventricle with a catheter across the aortic valve and secured in a fashion similar to method 500. The patch may be placed across the septal wall from the right ventricle. In this embodiment, a cannula is advanced into the right ventricle and a small hole may be made in the septum between the right and left ventricles. Another cannula with the properly shaped patch may be advanced into the right ventricle and through the hole in the septum where one half of the patch is deployed. The cannula may be pulled back into the right ventricle where the second half of the patch device is deployed. The deployment of the patch on both sides of the ventricle holds the patch securely in place.

In certain embodiments, a patch may be placed on the shaping device and introduced into the ventricle. The patch may be attached to the wall of the ventricle. A device on the patch may be tightened to cause the patch to reshape the ventricle over the shaping device. Once the desired shape is achieved the shaping device is removed and the patch left in place to hold the desired shape.

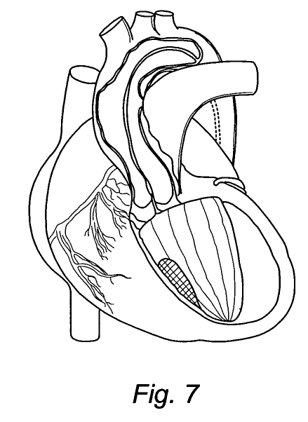

FIG. 6 depicts an embodiment for a method of reinforcing a dilated portion of an endocardial surface of a human heart. In this embodiment, a user preoperatively determines the location, size, and shape of the area of the septum to be reinforced. The user determines which appropriate reinforcing element will match the patient needs. Such reinforcing elements may be made from biocompatible materials and may take many forms. For instance, a patch material (discussed above) may be used. Such materials may be encapsulated within a deploying and securing mechanism that would allow them to be attached to the septal wall. An example of a reinforcing element may include a device made from shape memory metal that has the shape of the area to be reinforced and has biocompatible material covering the metal framework. As discussed above, the metal frame may be made of shape memory materials. The metal frame may provide a means to secure the material. The material may give substance to the metal frame to resist the pressure in the left ventricle. The reinforcing element may have radiopaque markings. Radiopaque markings may be positioned in a pattern that allows them to be viewed and analyzed postoperatively. The radiopaque markings may have a shape that matches the area to be reinforced. The reinforcing element may be shaped to match the patient anatomy and extent of injury. The securing device may have a mechanism by which the reinforcing element may be secured to the septum along the border zone between viable and non-viable tissue. In certain embodiments, the reinforcing element may have a first surface and a second surface. The first surface may be adapted to match the dilated portion of the endocardium. The second surface may be adapted to match an appropriate shape of the left ventricle. Thus, the reinforcing element may be used to reshape the ventricle.

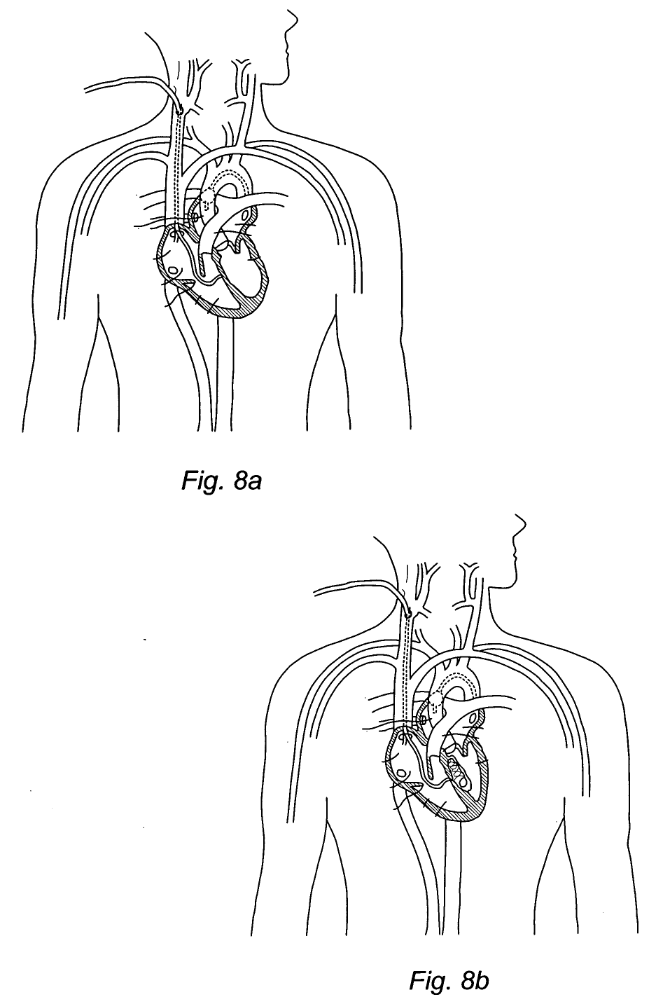

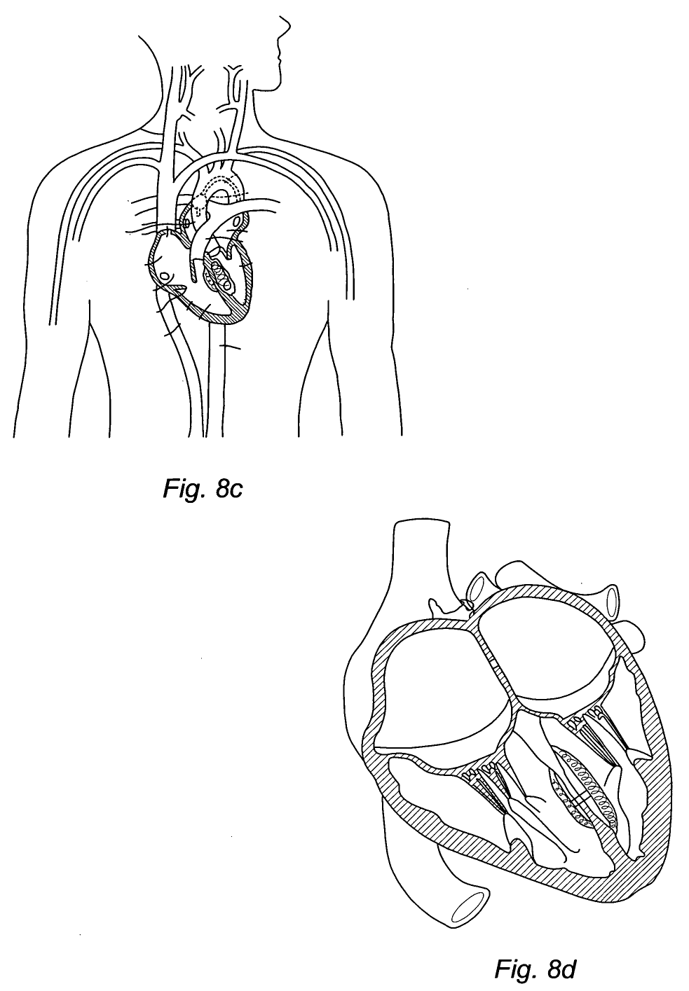

Turning back to FIG. 6, in 602, a user may insert a first catheter with a distal and proximal end percutaneously into a vasculature or vascular system (such as the jugular vein or the femoral vein) of the patient. The user may route a guidewire through a vein into the right ventricle in the vicinity of the area to be reinforced. With the guide wire in place, in 604, the surgeon may guide the first catheter into the right ventricle, as illustrated in FIG. 8a. Once the first catheter is in place, in 606, an incision may be made into the septal wall. In one embodiment, the incision may be accomplished with the aid of a trocar. The trocar may be advanced along the guide wire and positioned at the point in the septum that is generally the central point of the thinned septal region. The trocar may be pushed through the thinned septal wall to create a path between the right and left ventricles.

In 608, the guidewire may be advanced into the left ventricle from the right ventricle and, if a trocar is used, it may be withdrawn. In 610, a second catheter may be inserted over the guidewire such that the second catheter is introduced into the left ventricle. However, the second catheter may be coupled to a reinforcing element, as described above. In 612, the reinforcing element may be deployed in order to reinforce the portion of the endocardial surface, as illustrated in FIG. 8b. For instance, in the left ventricle side of the septum, one portion of the reinforcing element may be deployed with the edges of the device and securing mechanism resting on viable tissue of the septum at the border zone of the non-viable septal tissue. A second part of the securing mechanism may be deployed in the right ventricle and secured to the septal wall, as depicted in FIGS. 8c and 8d. A securing mechanism may be a type of mechanism used to occlude ventricular septal defects. NMT Medical (Massachusetts), W.L. Gore (Arizona) and AGA Medical Corporation (Minnesota) manufacture such devices. In 614, all components may be withdrawn from the right ventricle and the procedure is completed.

In certain embodiments, method 600 may include inserting a patch into the left ventricle using the reinforcing element. The patch may be positioned such that the patch aligns with a non-viable region in the heart. The reinforcing element may be expanded to an expanded shape. In the expanded shape the patch may be attached to the dilated portion of the heart. The expanding may anchor the reinforcing element to the septal wall in the right ventricle.

Some embodiments may include a reinforcing element and securing mechanism deploying on either side of the ventricle without creating a hole in the septum. In these embodiments a guidewire may be placed in the jugular or femoral veins and advanced to the proper location at the septum. The reinforcing element and securing mechanism may be advanced along the guidewire and the reinforcing element secured to the septum at the border zone of the non-viable septal tissue. The securing mechanism may be secured to the viable tissue at the edge of the border zone. An example of a type of securing mechanism that may be used are those similar to securing devices used to secure thoracic aortic aneurysm grafts. Medtronic (Minnesota), W. L. Gore (Arizona) and Boston Scientific (Massachusetts) make these securing mechanisms. The reinforcing element may be placed in the left ventricle side of the septum. At least one guidewire may be advanced through the aortic valve from the femoral artery or through one of the three great vessels coming off the aortic arch. The reinforcing element may be placed in a fashion similar to that used to place the device on the right ventricle side of the septum.

In some embodiments, two reinforcing elements may be positioned on either side of the septum in both the right and left ventricles without being connected through the septum. The placement of both reinforcing elements may be done as described for the individual placements in the right and left ventricles.

In certain embodiments, described procedures may be done as part of an endoscopic surgical ventricular repair, when the ventricle wall as well as the septum have been damaged due to ischemia. The placement of the reinforcing element may be as described in any one of the methods described herein. The endoscopic surgical ventricular repair procedure may include inserting a ventricular shaping device into the left ventricle via the femoral artery. A molding instrument may be inserted into the chest through a small opening in an intercostal space to reach the epicardium. This instrument may allow the user to press the ventricle wall against the shaping mandrel. Pressing the ventricle wall against the shaping mandrel may ensure that the ventricle is pressed against the mandrel. This pressing device may be withdrawn and a clasping instrument may be inserted. The molding instrument and clasping instrument may be one device. This device may take bites out of the ventricle wall starting at the edges of the area of non viable tissue that needs to be excluded to restore the ventricle to its correct shape, size, and/or contour. The bites may be made with suture type devices and/or clip type devices, for example. Such devices are currently used in an endoscopic surgery procedure and commonly referred to as GIA, in which a portion of the patient's stomach is excluded from the remainder of the stomach. These devices are manufactured by USSC (Connecticut), and Ethicon Endosurgery (Ohio). The clasping instrument may be partially closed to allow the user to ensure that he is properly shaping the ventricle onto the shaping mandrel. If the user determines that he has the clasping instrument placed properly, the device may allow for full closure. The implements placed by the clasping instrument when closed will have pulled the ventricle wall over the shaping mandrel and may maintain the ventricle's shape. Once the shaping is complete, the shaping mandrel may be collapsed and taken from the ventricle. Intraoperative imaging may be used during this procedure to aid the surgeon's view of the mandrel and ventricle interface.

If needed, revascularization during the beating heart method may be done either with stents alone or with a LIMA to LAD graft using a small thoracotomy and stents on any other vessel that need to be opened. All other aspects of surgical ventricular restoration may be performed.

The physiological changes a heart under goes after a major cardiovascular event (e.g., an infarction or heart attack) is well documented in textbooks. After an infarction, for example, the left ventricle undergoes contour change as a result of thinning and elongation of the infarcted region. This topographic alteration in the infarcted region may be referred as “Infarct Expansion” and/or “remodeling.” In about 30% of patients who suffer myocardial infarction or heart attack the remodeling is severe enough to develop heart failure. The remodeling process after a myocardial infarction is dependent on many factors including infarct size, infarct location, depth of infarction (transmurality), and/or ventricular wall stress. These factors may be clinically evaluated and patients with predilection to heart failure may be identified. However, there are not many options available to the doctors to prevent patients from slipping into heart failure.

Important factors in the remodeling process include increase in wall tension and wall stress in the areas surrounding the scar tissue. “Scar tissue” within the context of this application is generally defined to include damaged tissue (e.g., akinetic tissue and/or non viable tissue). Scar tissue may result from disease and/or major cardiac events (e.g., myocardial infarction). Wall tension may be generally defined as EQN 1:
Wall tension=(P×r)/2 (i.e., Law of Laplace). (1)
Where P is generally defined as pressure, and where r is generally defined as radius. Wall stress may be generally defined by EQN 2:
Wall Stress=Wall Tension/thickness=(P×r)/(2h). (2)
Where h is generally defined as wall thickness.

Thinning of the infarcted area (especially near the apex) may result in an increase in the radius by a factor of about 2 to 3 times. This increase in radius combined with the reduction of the wall thickness to ½ or ⅓ times the original wall thickness and an increase in ventricular filling pressure (due to increase in ventricular size) may result in an increase in wall stress 4 to 10 times pre-infarcted levels.

In some embodiments, an infarcted portion of a human heart may be reinforced. Reinforcement of an infarcted area may assist in substantially preserving a contour of the portion of the human heart. In certain embodiments, the portion of the heart may be a part of a ventricle (e.g., the left or right ventricle). Reinforcement of the portion of the heart may assist in preserving the radius of the ventricle that includes the portion of the heart. Preserving the radius of the portion of the heart will reduce wall stress. The reinforcing element may function to inhibit expansion of the volume of the ventricle (e.g., left and/or right ventricle). The reinforcing element may function to inhibit expansion of the interior wall/endocardial surface of the ventricle. In some embodiments, reinforcement of the portion of the heart may be accomplished by attaching a reinforcing element to the portion of the heart.

In some embodiments, a reinforcing element may inhibit expansion of an endocardial surface. The area of the endocardial surface may be averaged over a specified time period (e.g., a cardiac cycle) or phase. For example, the reinforcing element may be configured to inhibit expansion of an average of an endocardial surface over a cardiac cycle of the left or right ventricle. The reinforcing element may inhibit expansion of an endocardial surface while allowing substantially normal contraction and expansion of the heart during use. The reinforcing element may be configured to inhibit expansion of an endocardial surface such that normal contraction and expansion during a cardiac cycle of the heart remains substantially unimpeded. “Unimpeded” in the context of this application is generally defined as not slowed or prevented.

The reinforcing element may be attached to the interior wall (e.g., endocardial) surface of the portion of the heart. In some embodiments, a reinforcing element may be implanted as soon after a myocardial infarction occurs as is possible. The sooner the reinforcing element is positioned the more effectively it may prevent any further damage. In some embodiments, it may be desirable to implant a reinforcing element within a week of a myocardial infarction.

In many of the following figures and embodiments, reinforcement of the apex of the left ventricle will be discussed, serving merely in an illustrative manner. However, this reinforcement of the apex of the left ventricle should not be seen as a limiting embodiment, and reinforcement as described herein may be applied to any part of a human heart.

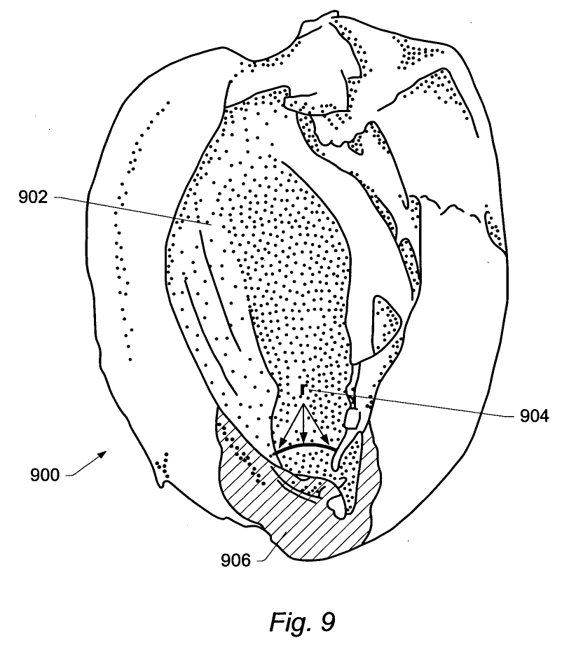

As depicted in FIG. 9, left ventricle 902 of human heart 900 has a certain shape (i.e., the contour varies). The radius of curvature at various points is different. Note that the radius 904 at the apex is the smallest; hence the apex may be particularly vulnerable to infarct expansion. FIG. 9 depicts an infarcted and/or non-viable portion 906 of left ventricle 902.

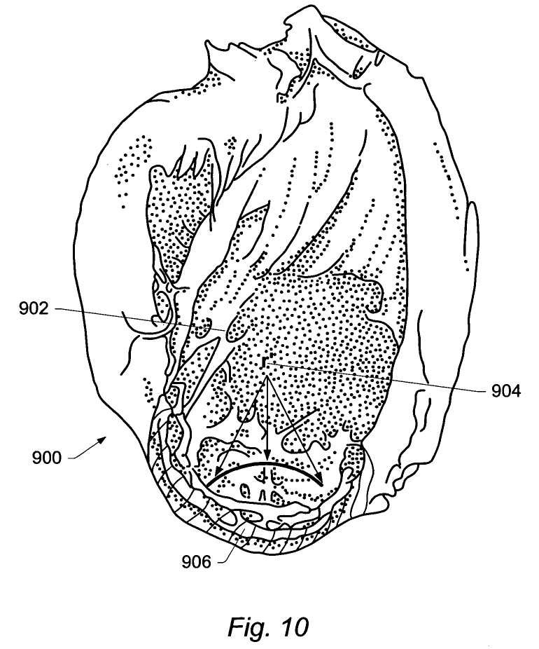

FIG. 10 depicts heart 900 after remodeling as a result of a myocardial infarction. Infarcted area 906 is depicted with the infarcted area thinned and the radius substantially increased. Thinning of infarcted area 906 and/or an increase in radius of the ventricle leads to an increase in wall stress. Also, the disfigured infarcted area 906 may result in global expansion of the ventricle. Note that radius 904 at the apex has increased and subsequently the volume of ventricular cavity 902 is substantially larger in FIG. 10 than in FIG. 9.

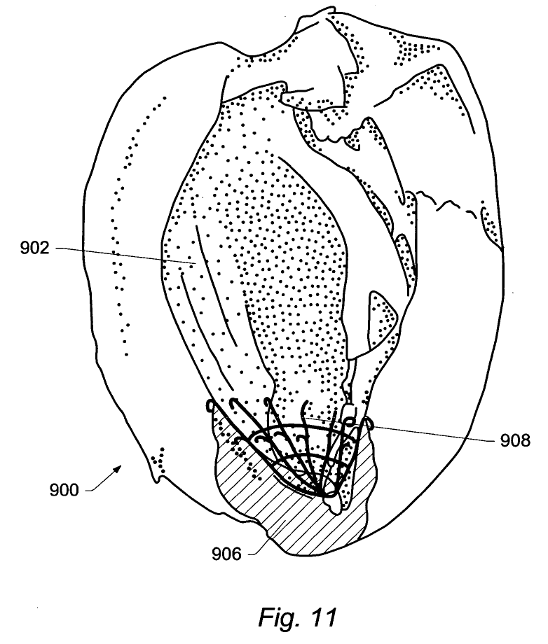

If reinforcing element 908 is implanted soon after infarction (as depicted in FIG. 11) the remodeling resulting from myocardial infarction may be inhibited and/or substantially lessened. In some embodiments, reinforcing element 908 may have a shape and/or size substantially mimicking infarcted region 906. In certain embodiments, reinforcing element 908 may be positioned in a ventricle soon after a myocardial infarction and before the ventricle has had an opportunity to begin remodeling itself; therefore, the reinforcing element may be of a shape and/or size of a portion of the existing ventricle. The size and/or shape of infarcted tissue may be obtained, for example, by analyzing delayed hyperenhanced cardiac MRI images. Cardiac analysis software such as SIMON (Chase Medical, Richardson, Tex.) is commercially available and able to perform such analysis. Cardiac analysis software, such as SIMON, may assist in analyzing the size and/or shape of the scar. Analysis software may assess the location of the scar with respect to other cardiac structures (e.g., papillary muscles, mitral valve, aortic valve, and/or septum).

In some embodiments, reinforcing element 908 may be specifically designed and manufactured according to patient specific requirements. In certain embodiments, reinforcing element 908 may be provided in a variety of predetermined shapes and/or sizes from which a user (e.g., a surgeon or doctor) may choose that which best suits the patients needs.

Reinforcing element 908 may include a plurality of coupling points. Coupling points may assist reinforcing element 908 to attach to a portion of the endocardial surface of a heart once the reinforcing element has been properly positioned. The portion of the endocardial surface may include non viable tissue (e.g., infarcted tissue). In some embodiments, a plurality of coupling points may assist a reinforcing element to releasably attach to an endocardial surface of a heart. Reinforcing elements, which are able to releasably attach to the endocardial surface of a heart, may allow a user to reposition the reinforcing element after initially positioning the reinforcing element. A user may choose to reposition the reinforcing element after having assessed the initial placement of the reinforcing element. The plurality of coupling points may be distributed over the “surface” of the predetermined shape of the reinforcing element. In some embodiments, the plurality of coupling points may be substantially evenly distributed over the surface of the reinforcing element.

Appropriate placement of a reinforcing element within the ventricle may allow the reinforcing element to provide structural support for at least the portion of the ventricle to which the reinforcing element is attached. Typically scar and/or non viable tissue resulting from a myocardial infarction may thin over time, consequently the radius along the endocardial boundary may be increased. Providing structural support to scar and/or non viable tissue may inhibit deformation of the ventricle. Deformation may include an increasing radius along the endocardial boundary. Deformation may be characterized by expansion of the endocardial surface of the heart and/or an increasing ventricular volume.

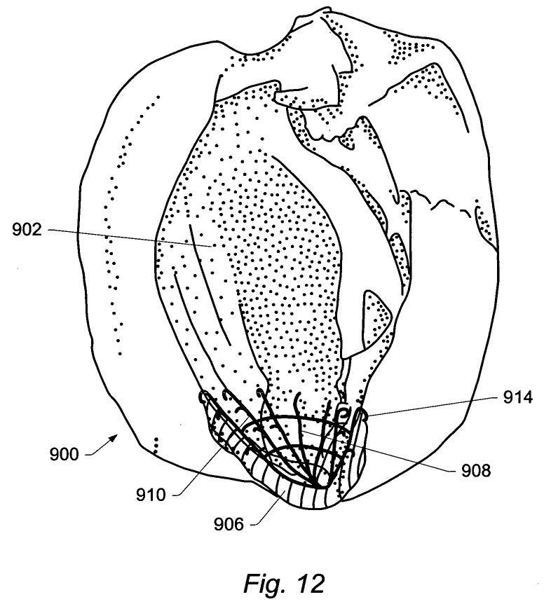

Inhibiting deformation of a ventricle may assist in preserving the endocardial radius, and, thus maintaining the wall tension. Wall stress may still increase relative to pre-infarction due to thinning of the wall; however, the increase in wall stress may be substantially less than if the reinforcing element were not in place. With a reinforcing element in position, wall thinning may also be reduced. As non viable tissue thins, outer wall tissue may be pulled up towards the reinforcing element, due at least in part to the inner wall being attached to the reinforcing element inhibiting the inner wall from expanding outward. Outer wall tissue pulled towards the reinforcing element may include viable tissue in the border (an example of this is depicted in FIG. 12). This redistribution of tissue, in response to positioning the reinforcing element, may result in substantially thicker wall tissue in the infarcted region compared to if the reinforcing element were not used.

With no change in wall tension and reduced wall thinning, ventricular wall stress may not be significantly increased. Therapy, including the use of a reinforcing element or a reinforcing element in combination with the use of known cardiac drugs (e.g., diuretics, calcium channel blockers (all of which reduce the load on the heart)) and/or revascularization (bypass or stent), may prevent patients from slipping into heart failure.

In some embodiments, reinforcing element 908 may include a “mesh basket” as depicted in FIG. 13. Reinforcing element 908 may be formed at least in part by a plurality of conduits 910. Conduits 910 may be formed at least partially of shape memory metals, properties of which are described herein. A specific example of a shape memory metal from which conduits may be formed includes, but is not limited to, nitinol. In some embodiments, a reinforcing element may be formed from any biocompatible material (or any material which may be adapted to be biocompatible) that may offer increased structural strength and/or integrity to a portion of the heart to which the reinforcing element is attached.

In some embodiments, a reinforcing element may be formed from materials (e.g., shape memory metals) allowing the reinforcing element to take on one or more shapes. The materials may be flexible, and “remember” at least one predetermined (e.g., a predetermined second shape). However, the flexibility of the material may allow the reinforcing element to assume other shapes, other than the predetermined second shape. The second predetermined shape may substantially emulate a size and/or shape of at least a portion of an endocardial surface of a ventricle. The reinforcing element may be able to form a first predetermined shape. A diameter of the second predetermined shape is larger than a diameter of the first predetermined shape. Diameter in this particular instance refers to an average diameter along a central axis. The first predetermined shape may allow the reinforcing element to navigate through a relatively confined space (e.g., relative to a ventricle) such as, for example, a catheter, a cannula, and/or a vasculature system of a human body.

In certain embodiments, reinforcing element 908 has conduits 910 extending to the periphery of the scar, as depicted in FIG. 12. In some embodiments, conduits 910 may be of various lengths. Conduits of various lengths may engage a portion of a heart at various points other than at the periphery of the portion. In general, the more coupling points the better reinforcement of the portion of the heart. Larger portions of infarcted tissue may require more coupling points than relatively smaller portions of infarcted or non viable tissue.

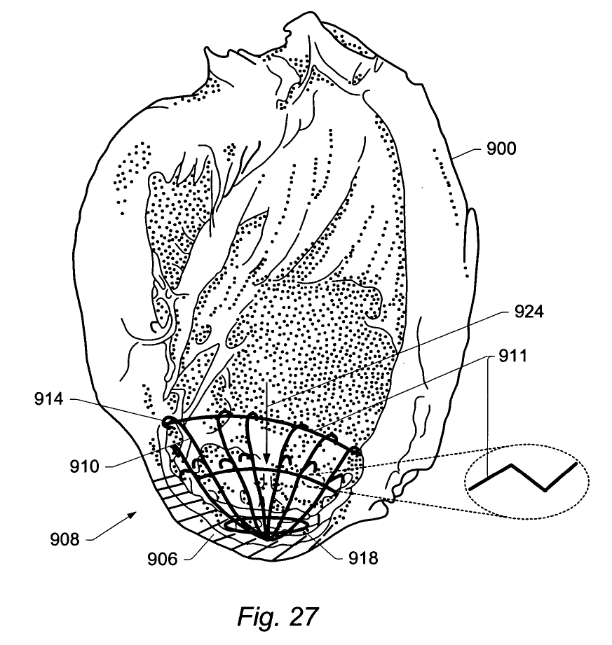

In some embodiments, reinforcing element 908 may include one or more support elements 911, as depicted in FIG. 27. One or more support elements 911 may couple or attach together two or more of conduits 910. Support elements 911 may inhibit the reinforcing element from expanding beyond a second predetermined shape during use. Support elements 911 may allow the reinforcing element to contract to an extent that allows the heart to operate normally. In some embodiments, support elements 911 may include an accordion style embodiment (as depicted in FIG. 27) to allow appropriate contraction of a heart as well as to substantially inhibit expansion of a reinforcing element beyond a second predetermined shape.

In some embodiments, a reinforcing element may include a mechanism for attaching to or engaging a surface (e.g., endocardial) of a heart. A coupling mechanism may include any mechanism that upon activation effectively couples the reinforcing element to a portion of human tissue (e.g., cardiac tissue). The coupling mechanism may releasably attach to cardiac tissue, allowing a user to reposition the reinforcing element after initially positioning and coupling the reinforcing element to a portion of the heart.

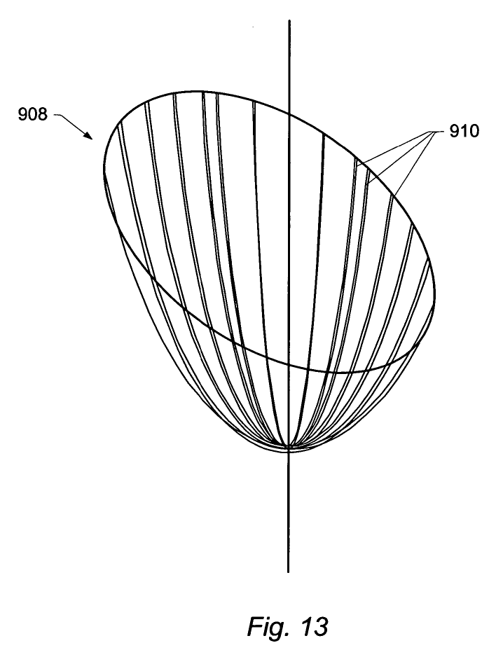

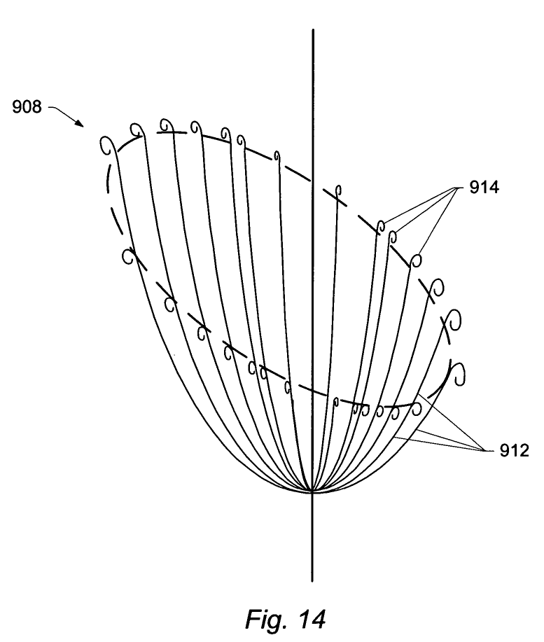

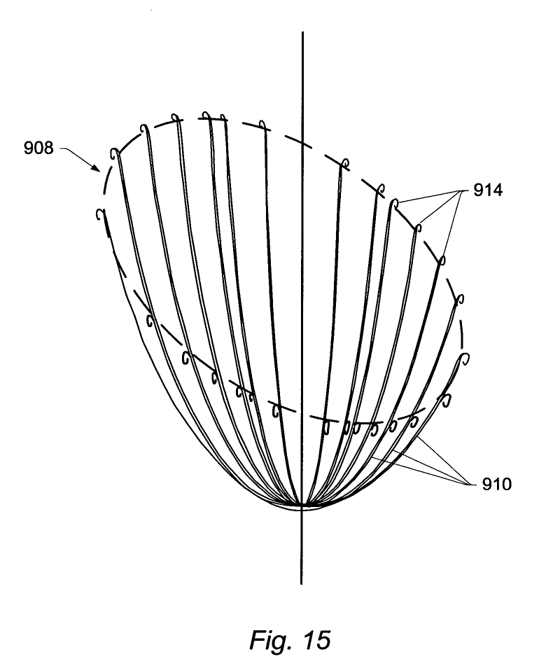

In some embodiments, a coupling mechanism may include a plurality of elongated members 912, as depicted in FIG. 14. FIG. 14 does not depict a plurality of conduits 910 for purposes of clarity. Elongated members 912 may be at least in part formed from shape memory metals (e.g., nitinol). In some embodiments, elongated members 912 may include pointed, sharp, and/or needle tipped distal ends 914. Elongated members 912 may be positioned in conduits 910 (depicted in FIG. 13). One or more elongated members may be positioned in the conduits. In certain embodiments, only one elongated member may be positioned in each conduit. When in a retracted or inactivated state, at least distal ends 914 may be positioned substantially in conduits 910. When in an extended or activated state, at least distal ends 914 may be extended substantially beyond the distal end of each respective conduit 910. Distal ends 914 of elongated members 912 may be predisposed (e.g., preshaped in the case of shape memory metal based elongated members) to exist in a substantially inwardly curled state as depicted in FIG. 14. FIG. 15 depicts reinforcing element 908 with distal ends 914 of the elongated members activated and extended from conduits 910.

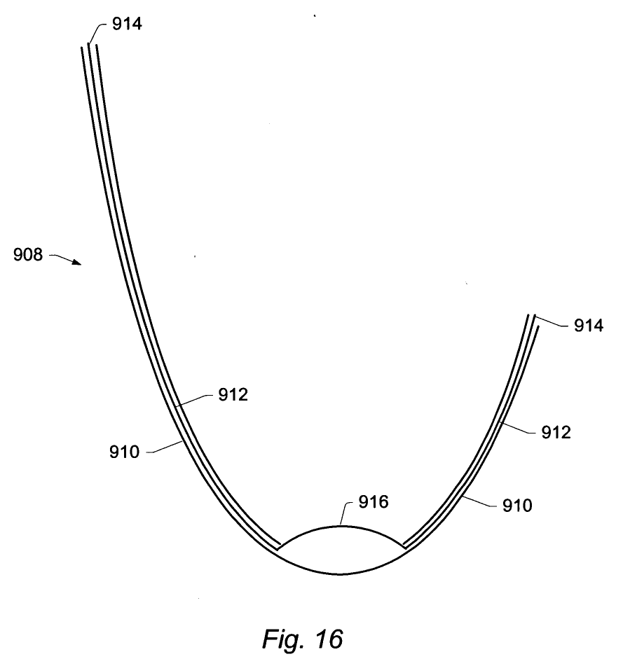

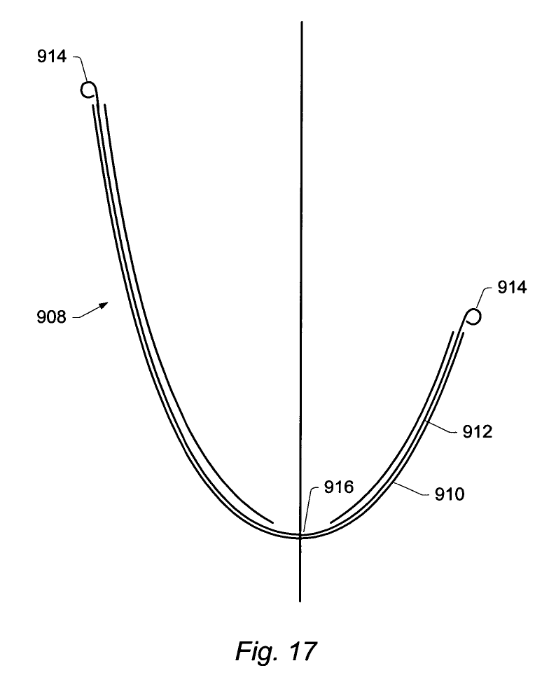

FIG. 16 depicts a cross sectional view of a portion of reinforcing element 908, including one conduit 910 and one elongated member 912. Elongated member 912 is depicted in a retracted/inactivated state in FIG. 16. Elongated member 912 may be formed from a substantially flexible material such that the distal end 914 may bend and flex so as to be positionable in conduit 910 when in a retracted state. When elongated member 912 is in an extended/activated state distal end 914 may change shape to a substantially inwardly curled state (as depicted in FIG. 17). Distal ends 914 in the substantially curled state may pierce tissue (when positioned in a portion of a heart) attaching reinforcing element 908 to the portion of the heart.

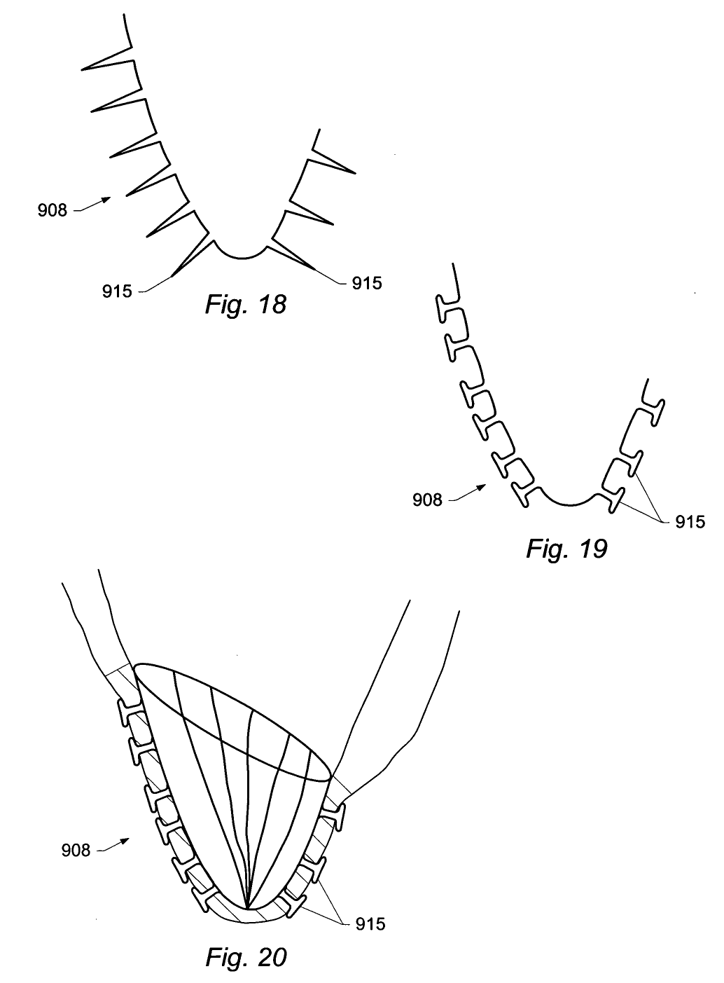

In some embodiments, a reinforcing element may include a coupling mechanism. FIG. 18 depicts an embodiment of a portion of reinforcing element 908 with a coupling mechanism in an inactivated/disengaged state. The coupling mechanism may include coupling portions 915. Coupling portions 915 may be attached to a portion of reinforcing element 908 and/or the coupling portions may be formed as part of a portion of the reinforcing element (as depicted in FIG. 18). In embodiments of a reinforcing element including coupling portions, conduits may not be necessary and a wire cage forming the reinforcing element may be formed from substantially solid elongated members. In some embodiments, coupling portions may be formed from a substantially flexible material facilitating their passage through a constricted passage such as a flexible conduit. A flexible material may allow the coupling portions to bend so as to allow the reinforcing element to pass through a conduit. Coupling portions may include a first form as depicted in FIG. 18 The first form may facilitate penetration of human tissue.

FIG. 19 depicts an embodiment of a portion of reinforcing element 908 with a coupling mechanism including coupling portions in an activated/engaged state. Upon penetrating tissue coupling portions 915 may be activated and change shape. Coupling portions may change shape into a second form as depicted in FIG. 19. The second form may inhibit extraction of the coupling portions from penetrated tissue, effectively attaching the reinforcing element to an endocardial surface of a heart.

In some embodiments, coupling portions may be formed from shape memory metals. Shape memory alloys have a unique property of shape retention at different states. The material exists at two forms i.e. Austenite and Martensite. For example a wireform may be formed into a particular shape in an Austenite state and deformed i.e. stretched to make it straight, this deformation process will convert the wireform into its Martensite state. However, when the wire is heated above a transformation temperature it goes back to its Austenite state, and recovers its original shape. A reversible solid-state phase transformation from austenite to martensite occurs, for example, on cooling (or by deformation) and the reverse transformation from martensite to austenite occurs, for example, on heating (or upon release of deformation). The transformation temperature is dependent on the material composition. Materials can be engineered to have transformation temperatures just a few degrees above body temperature (e.g., 45° C.). There are many companies, which specialize in nitinol fabrication like Memry Corporation (www.memry.com), Nitinol Devices and Components (www.Nitinol.com).

In some embodiments, coupling portions may be activated by subjecting them to mild heat to transform into a second form as depicted in, for example, FIG. 19. The heating of the device can be accomplished by passing low powered electricity through the device, which will generate resistance heating in the device.

Heat generated (∝) may be expressed in the equation: I² R. Where I is Current, and R is electrical resistance of the device/material.

In some embodiments, distal ends of coupling portions may be positioned substantially in an endocardial surface of a ventricle upon positioning a reinforcing element. Once positioned one or more of the coupling portions may be activated changing the shape of the activated coupling portion from a first form to a second form. The second form may inhibit extraction of the distal ends of the activated coupling portions from the endocardial surface of the ventricle. FIG. 20 depicts an embodiment of a portion of reinforcing element 908 with a coupling mechanism in an activated/engaged state positioned in a left ventricle of a heart wherein portions 915 of the coupling mechanism extend partially into an endocardial surface surface.

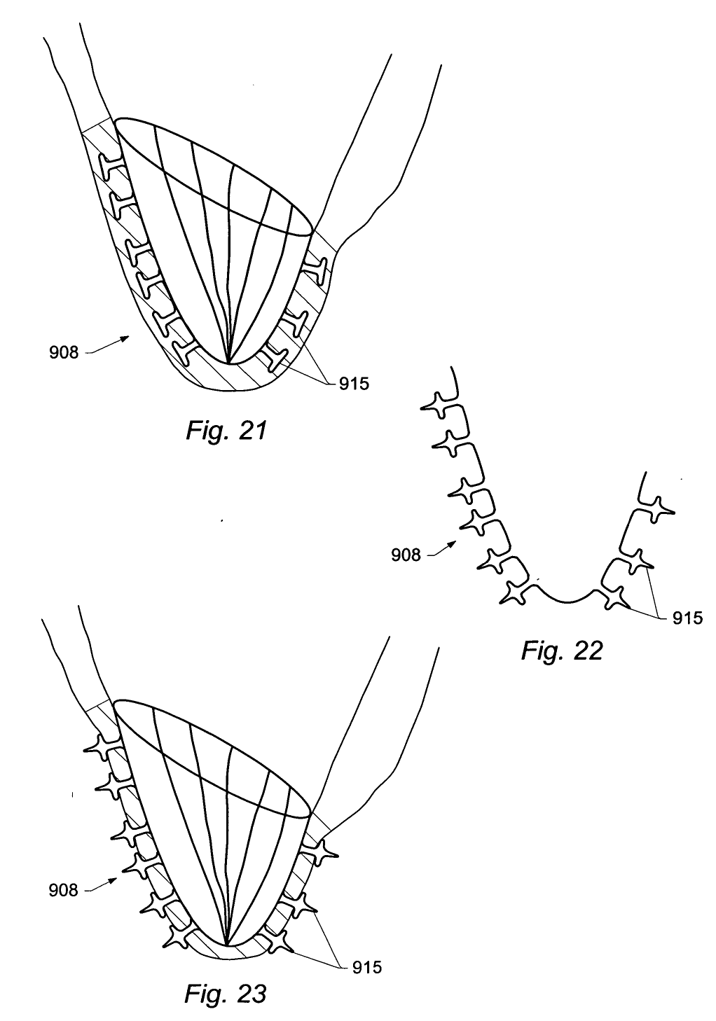

In other embodiments, distal ends of coupling portions may be extended substantially through an endocardial wall of a ventricle upon positioning a reinforcing element. Once positioned one or more of the coupling portions may be activated changing the shape of the activated coupling portion from a first form to a second form. The second form may inhibit extraction of the distal ends of the activated coupling portions through the endocardial wall of the ventricle. FIG. 21 depicts an embodiment of a portion of reinforcing element 908 with a coupling mechanism in an activated/engaged state positioned in a left ventricle of a heart wherein portions 915 of the coupling mechanism extend through an endocardial wall surface.

In some embodiments, a coupling mechanism may include coupling portions. Distal ends of coupling portions may include broadened parts or “barbs”. FIG. 22 depicts an embodiment of a portion of reinforcing element 908 with a coupling mechanism including coupling portions 915 with barbs. Barbs may make disengagement of the reinforcing element difficult. FIG. 23 depicts an embodiment of a portion of reinforcing element 908 with a coupling mechanism positioned in a left ventricle of a heart wherein portions 915 of the coupling mechanism extend through an endocardial wall surface.

In some embodiments, activation mechanism 916 (depicted in FIGS. 16 and 17) may be used to activate reinforcing element 908 during use. Activation mechanism 916 may include any mechanism which, when employed after the reinforcing element is positioned, sets in motion a coupling mechanism (e.g., such as elongated members 912 described herein) that attaches the reinforcing element to surrounding tissue.

Some embodiments may include an activation mechanism such as is depicted in FIGS. 16 and 17. Activation mechanism 916 may include a center region. The center region may function to couple at least two elongated members 912. The activation mechanism may be formed by coupling at least some of the proximal ends of the elongated members to each other. The activation mechanism may be formed by coupling at least two of the conduits to the center region. The conduits coupled to the center region may radiate out from the center region. In an inactivated state, as depicted in FIG. 16, the activation mechanism may include portions of elongated members 912 forming a convex shape. Activation mechanism 916 may be activated by applying pressure and inverting the convex shape to a concave shape. Inverting the convex shape to a concave shape pushes elongated members 912 further in and through conduits 910, extending distal ends 914 beyond the. distal ends of the conduits.

In some embodiments, activation mechanism 916 may include a center region. The center region may include an opening. For example, the activation mechanism may include a substantially circular ring. Proximal ends of elongated members 912 may be coupled to the periphery of the circular ring. Activation mechanism 916 may be activated by applying pressure to the ring and inverting the convex shape to a concave shape. The opening may allow a guidewire to pass through reinforcing element 908. A guidewire may assist in positioning the reinforcing element during surgery.

In some embodiments, a reinforcing element may be made of a preshaped patch material, with a shape and/or size similar to an infarcted area. In certain embodiments, a patch may be delivered over a shaper (e.g., a wire mesh mandrel). Once the patch is placed at desired location, the patch may be attached to the scar by injecting biocompatible glue between the patch and scar tissue.

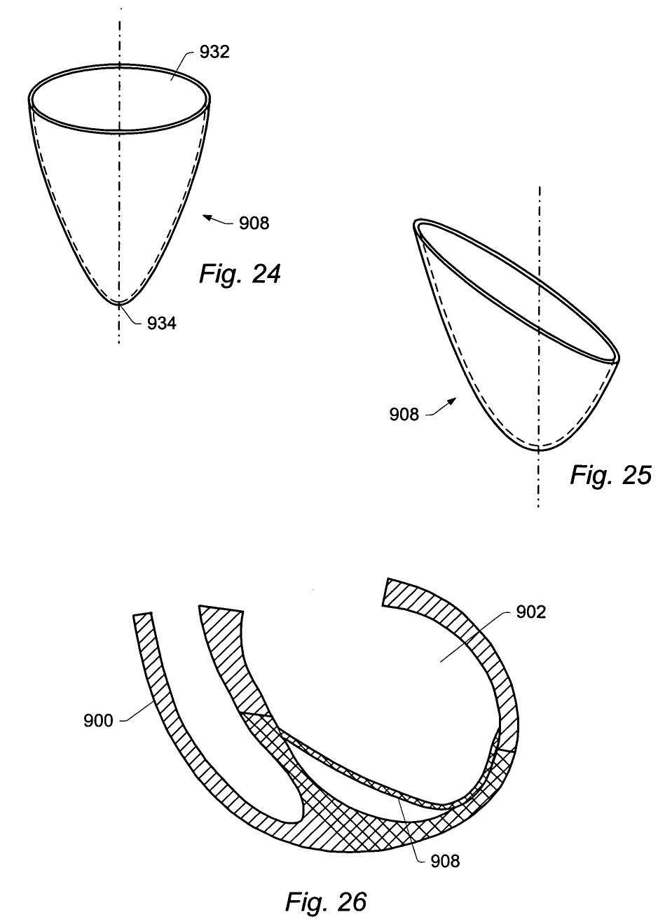

FIG. 24 depicts an embodiment of reinforcing element 908 which may be “cone shaped” similar to the lower portion of the left ventricle. This conical shape may be open at base 932 of the patch and have tip 934, which may function as a new apex after insertion into the patient.

In some embodiments, a tissue contact surface of a reinforcing element may be partially or completely concave, convex, flat, or a combination.

In some embodiments, a reinforcing element may be made and sold in a selection of stock sizes (e.g., based on size, ventricular volume reduction, distance from the ventricular septal apex, and/or other basis). The reinforcing element may be formed according to a particular patient using the Chase Medical SIMON system, and or any other suitable imaging and or evaluation means.

In some embodiments, a reinforcing element may include at least one suture, wire, rod, bar, band, sheet, ribbon, combination or other suitable structural device. Structural devices may be formed from shape memory or superelastic material (e.g., Nitinol), polymer, metal, metal alloy, or combination of the above materials, and/or any suitable material.

In some embodiments, a reinforcing element may have at least one hole, slot, groove, combination or other, at one or more locations, to modify/effect the flexibility, elasticity, or compression force of the reinforcing element.

In some embodiments, a reinforcing element may include, as a separate component, and/or a coating. The component or coating may be formed from barium sulfate, bismuth trioxide, tantalum, platinum, gold, a combination of the above, or other radiopaque substance. The component or coating may increase the visibility using fluoroscopy, and or to be used as a marker and or reference points.

In one embodiment, three-dimensional reinforcing element 908 may be made out of a single piece of material to make the patch seamless. Reinforcing element 908 may come in different sizes. The sizes may correspond to a size of a shaping device or to the size of an appropriate ventricle. Reinforcing element 908 may be easily cut, so that surgeons may match the material to the akinetic area of the patients heart. Such a modified reinforcing element is illustrated in FIG. 25.

Reinforcing element 908 may be made from a polyester, for example a Dacron™ like material. Such material is currently used for implantable prosthetics. Woven materials, such as polyesters, may be impregnated with materials that function to inhibit penetration of fluids such as blood. Materials which may be impregnated to inhibit leaking may include collagen. Reinforcing element 908 may be manufactured in a variety of methods known in textile manufacturing (e.g., bonding, weaving, cut to shape, sewing, etc.). An embodiment may have a three dimensional patch made from ePTFE (expanded polytetrafluoroethylene). In some embodiments, reinforcing element 908 may be formed from bioprosthetic materials. Examples of bioprosthetic materials may include, but are not limited to, porcine cells and bovine cells.

In some embodiments, reinforcing element 908 may be made of synthetic material that, when subjected to a stimulus, flexes or contracts. Synthetic material which flexes/contracts in response to stimuli may aid in contraction of the left ventricle. In these embodiments, reinforcing element 908 may be made out of ion exchange material. Ion exchange material may be coated with a noble metal, shape memory metals, and/or electrosensitive gels that change shape in reaction to an electrical signal. Such a patch could be simulated to flex in synchronization with the cardiac cycle of a pacemaker or other implantable controller. A controller may be programmed transcutaneously and the amount of contraction may be controlled and adjusted. An energy source of the electric signal may come from a rechargeable battery that may be charged transcutaneously.

An embodiment may have reinforcing element 908 made totally from biologic material that contracts and assists in the contraction of the ventricle. Such an embodiment may be made from autologous cells, xeno transplant, cultured skeletal muscle cells, cultured bone marrow cells, cultured cardiac muscle cells, and/or cultured smooth muscle cells. A growth factor to stimulate tissue growth may be impregnated in the biologic material to be released over time.

Reinforcing element 908 may also be a structure that is impregnated with biological material that contracts and assists in the contraction of the ventricle. The structure could be impregnated with skeletal muscle cells, bone marrow cells, cardiac muscle cells, and/or smooth muscle cells. The structure may be bioabsorable and may be absorbed by the body over time, leaving only the biological material. In an embodiment, a growth factor may be impregnated in the structure to be released over time.

A reinforcing element may provide structural support for at least a portion of a ventricle to which the reinforcing element is attached by appropriate placement of the reinforcing element in the ventricle. Typically scar and/or non viable tissue resulting from a myocardial infarction thins over time, consequently the radius along the endocardial boundary may increase. Providing structural support to scar and/or non viable tissue may inhibit deformation of the ventricle. Deformation may include an increased radius along the endocardial boundary. Inhibiting deformation of a ventricle may assist in preserving the endocardial radius, and, thus maintaining the wall tension.

Certain embodiments of a reinforcing element inhibit further deformation of a ventricle. Methodology and apparatus embodiments that inhibit further deformation of a ventricle require a user to position a reinforcing element as soon as is feasible after a cardiovascular event (e.g., a myocardial infarction) in a subject. The possibility of ventricular deformation is lessened by positioning a reinforcing element in a ventricle as early as possible. Early positioning of the reinforcing element may increase the beneficial impact of the reinforcing element in inhibiting further deformation. Inhibiting further deformation may not be enough, however, in cases where moderate to extensive ventricular deformation has already occurred.

In some embodiments, a reinforcing element may include an adjustment mechanism. The adjustment mechanism may facilitate changing a dimension of one or more portions of the reinforcing element when activated. “Dimension” within the context of this application generally includes, for example, such concepts as length, width, radius, diameter and/or volume. The adjustment mechanism may be used to change the dimension of one or more portions of the reinforcing element when the reinforcing element is attached to a portion of a ventricle. Using the adjustment mechanism to change the dimension of one or more portions of the reinforcing element may result in a change in the dimension of the portion of the ventricle. FIG. 27 depicts an embodiment of reinforcing element 908 including adjustment mechanism 918. The adjustment mechanism is in an inactivated state. The reinforcing element and adjustment mechanism are positioned in a left ventricle of a human heart.

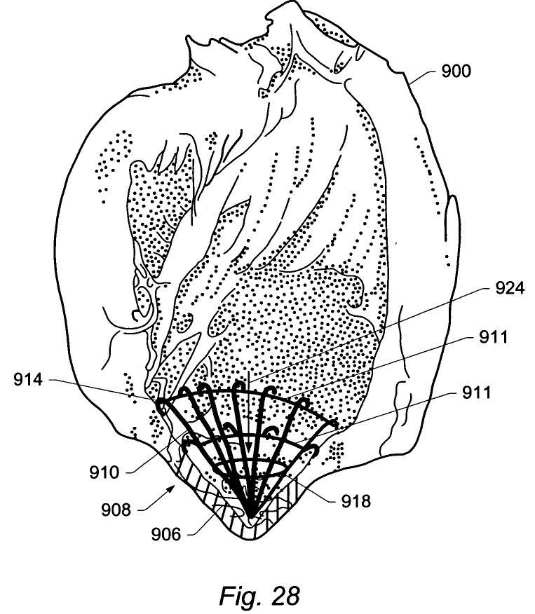

FIG. 28 depicts an embodiment of reinforcing element 908 including adjustment mechanism 918 in an activated state. Upon attaching the reinforcing element to a portion of an interior/endocardial surface of a ventricle, the adjustment mechanism may be activated. In some embodiments, activating an adjustment mechanism may reduce the diameter/radius of the reinforcing element and consequently reduce the diameter/radius of the portion of the ventricle. Reduction of a diameter of a portion of an enlarged ventricle may reduce the diameter of the portion to a pre-enlarged diameter.

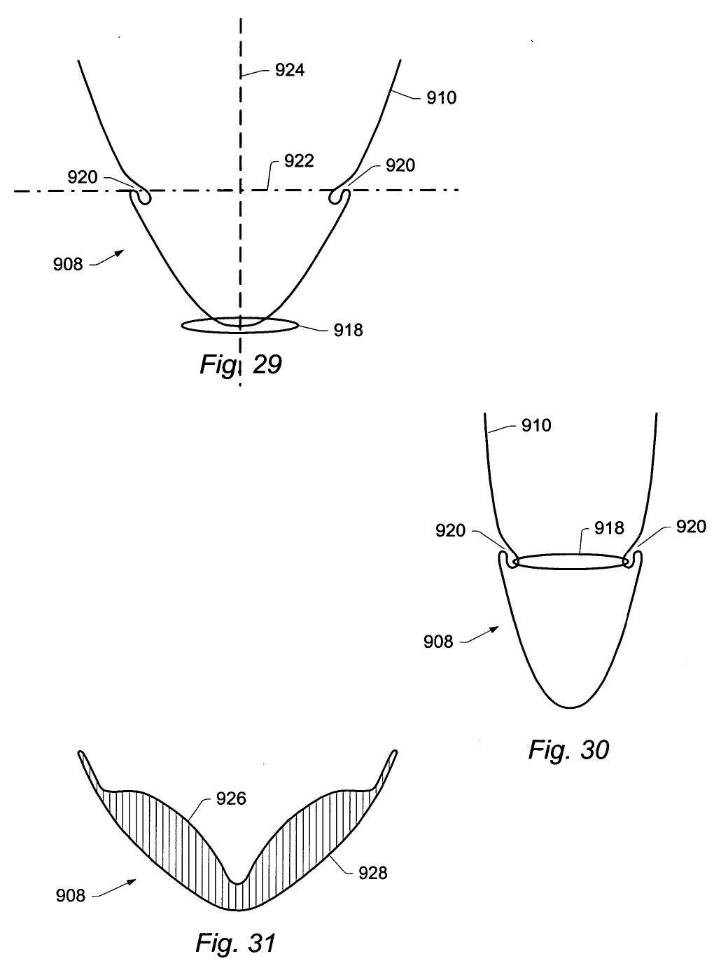

In some embodiments, a reinforcing element may include an engagement mechanism. FIG. 29 depicts an embodiment of a portion of reinforcing element 908, adjustment mechanism 918, and engagement mechanism 920, including a sectional view of the reinforcing element with the adjustment mechanism in an inactivated/disengaged position. FIG. 30 depicts an embodiment of a portion of reinforcing element 908, adjustment mechanism 918, and an engagement mechanism 920, including a sectional view of the reinforcing element with the adjustment mechanism in an activated/engaged position. Engagement mechanism 920 may spatially secure adjustment mechanism 918 after a user has activated and properly positioned the adjustment mechanism. In some embodiments, a position of engagement mechanism 920 may be manufactured based upon patient specific requirements. The position of engagement mechanism 920 may, in combination with the dimension of adjustment mechanism 918, determine the adjusted dimension of reinforcing element 908 (and consequently of any portion of a ventricle attached to the reinforcing element). Adjustment mechanism 918 may come in a variety of premanufactured dimensions and/or be manufactured for a specific patient's needs.

In certain embodiments, reinforcing element 908 may include a plurality of engagement mechanisms 920. Engagement mechanisms 920 may be formed as part of and/or coupled to the outer perimeter of portions of reinforcing element 908. In some embodiments, reinforcing element 908 may include at least two engagement mechanisms 920. The engagement mechanisms may be positioned substantially equidistant around the outer perimeter of the reinforcing element. The engagement mechanisms may be positioned substantially within plane 922 oriented substantially perpendicular to central axis 924 (as depicted in FIG. 29) of the deployed reinforcing element.

In some embodiments, a plurality of engagement mechanisms 920 may be grouped into multiple sets or groups. Each set or group may be positioned substantially within a plane oriented substantially perpendicular to a central axis of the deployed reinforcing element. Each set or group may be positioned at different elevations relative to one another on the deployed reinforcing element. Using sets of engagement mechanisms in combination with sets of adjustment mechanisms with different dimensions may allow a user to more accurately fit reinforcing element 908 to a specific subject or patient. By adjusting the height of adjustment mechanism 918 relative to deployed reinforcing element 908, a shape of a ventricle may be adjusted.

In some embodiments, coupling mechanisms (for example, as depicted in FIGS. 16 and 17) may not be positioned between an apex of reinforcing element 908 and engagement mechanisms 920. Advantages of an absence of coupling mechanisms between reinforcing element 908 and engagement mechanisms 920 may include not inhibiting the activation of adjustment mechanism 918, which may be positioned adjacent the apex of the reinforcing element prior to activation. In some embodiments, engagement mechanisms 920 may be positioned between an apex of reinforcing element 908 and about 50% of the total height of the reinforcing element. In some embodiments, engagement mechanisms 920 may be positioned between about 30% and about 50% of the total height of reinforcing element 908, measuring from an apex of the reinforcing element.

In some embodiments, engagement mechanisms 920 may include rings or depressions formed in one or more conduits 910 as depicted in FIGS. 29 and 30. Upon deploying and positioning reinforcing element 908 in a ventricle, adjustment mechanism 918 may initially be in an inactivated state, as depicted in FIG. 29. Upon attaching the reinforcing element to the endocardial surface of a heart (e.g., a ventricle), a user may activate the adjustment mechanism by positioning one or more portions of the adjustment mechanism in the engagement mechanisms, as depicted in FIG. 30. Activating the adjustment mechanism may reshape and/or adjust the volume of the enlarged ventricle so that the newly reshaped ventricle substantially mimics the preenlarged shape of the ventricle (e.g., the shape of the ventricle before a myocardial infarction).

FIGS. 29 and 30 depict one embodiment of engagement mechanisms 920. In some embodiments, engagement mechanisms may be any mechanism that will substantially inhibit undesirable movement of an adjustment mechanism once the adjustment mechanism has been positioned by a user. In certain embodiments, for example, engagement mechanisms 920 may be coupled to one or more conduits 910 of reinforcing element 908. The engagement mechanisms may be positionable along at least a portion of the conduit to allow a user to adjust the activated position of the engagement mechanism relative to the deployed reinforcing element.

In some embodiments, an adjustment mechanism may be coupled to a reinforcing element before activation. Attaching the adjustment mechanism to the reinforcing element may facilitate proper placement of the adjustment mechanism in a ventricle relative to the position of the reinforcing element. The adjustment mechanism may be releasably attached to the reinforcing element. Releasably attaching the adjustment mechanism to the reinforcing element may combine the benefits of an attached adjustment mechanism (e.g., facilitating initial placement) and a non-attached adjustment mechanism (e.g., freedom of movement relative to the reinforcing element during an adjustment stage).

In some embodiments, an adjustment mechanism may be attached to a reinforcing element with an elongated member. The elongated member may be formed from biocompatible materials. The elongated member may be of a length to allow the elongated member to move freely enough to perform its function of adjusting the dimension of one or more portions of the reinforcing element. In some embodiments, an elongated member may be detachable from an adjustment mechanism and/or a reinforcing element. In some embodiments, an elongated member may be formed from a biocompatible material that dissolves over time. The biocompatible material may last until all placement and/or adjustment of the dimension of a reinforcing element is completed.

In some embodiments, one or more portions of a reinforcing element may be formed from a biocompatible material that dissolves over time. For example, all of a reinforcing element may be formed from dissolvable biocompatible materials, except for an adjustment mechanism. Over time an adjustment mechanism may naturally couple to surrounding cardiac tissue as the remainder of the reinforcing element dissolves, such that the adjustment mechanism eventually functions as the reinforcing element (e.g., inhibiting deformation of an interior chamber of a human heart).

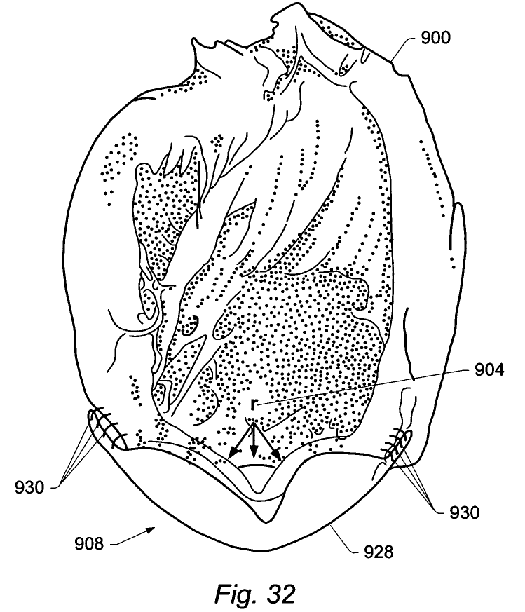

In some embodiments, a reinforcing element may be attached to a portion of an external surface of a heart (e.g., epicardial). FIG. 31 depicts an embodiment of an externally placed reinforcing element 908. FIG. 32 depicts an embodiment of externally placed reinforcing element 908 attached to a portion of a human heart during use. Externally placed reinforcing element 908 may be attached to a portion of an external surface of a human heart. In some embodiments, reinforcing element 908 may be attached to an epicardial surface of an apex of a left ventricle of a human heart. Reinforcing element 908 may be a three-dimensional object. Reinforcing element 908 may be preshaped from biocompatible materials that substantially inhibit deformation of the reinforcing element. Reinforcing element 908 may include inner surface 926 and outer surface 928, as depicted in FIG. 31. Inner surface 926 may reshape a portion of an interior chamber of a human heart during use. Reshaping the portion of an interior chamber may include changing (e.g., reducing) a volume of the interior chamber. The portion of the interior chamber may substantially adapt to a contour of inner surface 926. In some embodiments, reshaping an interior chamber of a heart with reinforcing element 908 may include restoring an enlarged ventricle to a substantially pre-enlarged state.

Reinforcing element 908 may be attached to a portion of an external surface of a heart using any method known to one skilled in the art. In some embodiments, sutures 930 may be employed to attach reinforcing element 908 to a portion of an exterior surface of a heart, as depicted in FIG. 32. In some embodiments, biocompatible glue may be employed to attach a reinforcing element to a portion of an exterior surface of a heart. In some embodiments, staples may be employed to couple a reinforcing element to a portion of an exterior surface of a heart.

In some embodiments, sutures may be employed to couple a reinforcement element to a surface of a heart. Sutures may be applied manually by hand. Sutures may be applied using assistance, such as that provided by an auto suture device (e.g., Sutura SuperStich).

In some embodiments, biocompatible glues or adhesives may be used to couple a reinforcing element to a surface of a heart. The reinforcing element (e.g., patch) may have a biocompatible contact adhesive or other material to bond or secure the reinforcing element to the target tissue. Adhesives may be used to secure, or assist in securing together layers of a reinforcing element. The adhesive/bonding compounds/solutions may be added during the manufacturing process, just prior to deployment, and/or after the reinforcing element has been deployed. Bonding materials may be in the form of a liquid, semi solid, and/or solid.

Suitable bonding materials may include gels, foams and/or microporous mesh. Suitable adhesives may include acrylates, cyanoacrylates, epoxies, fibrin-based adhesives, other biological based adhesives, UV light and/or heat activated and/or other specialized adhesives. The adhesive may bond on initial contact, or take longer to bond, to allow repositioning if desired.

In some embodiments, an adhesive or glue may include a crystalline polymer that changes from a non-tacky crystalline state to an adhesive gel state when the temperature is raised from room temperature to body temperature. One example of such a material is available under the trade name Intillemer™ adhesive, available from Landec Corp. as well as composites and combinations thereof and combinations of other materials. Suppliers of surgical adhesives include, but aren't limited to, Plasto (Dijon, France), Haemacure (Montreal, Canada), Cohesion (Palo Alto, Calif.), Cryolife (Kennesaw, Ga.), TissueLink (Dover, N.H.), and others.

To increase the work time of the adhesive or allow repositioning of the device after it has been deployed, the adhesive may be blended with a material, such as a starch or other material, that dissolves and retards or delays bonding to allow repositioning of the device after it has been deployed. A degradable coating can be placed over the adhesive coating so that it degrades and exposes the adhesive to the target tissue.

In some embodiments, adhesives or glues may include hydrophilic gels (hydrogels), foams, gelatins, regenerated cellulose, polyethylene vinyl acetate (PEVA), as well as composites and combinations thereof and combinations of other biocompatible swellable or expandable materials.

In some embodiments, securing mechanisms may be employed to secure a reinforcing element to a surface of a heart. Securing mechanisms or anchors may inhibit movement and/or dislodgement upon being deployed into tissue. Securing mechanisms may include anchoring features. Anchoring features may include barbed anchor features. Anchoring features may include coiled anchor features. Anchoring features may assist securing mechanisms to inhibit movement of a reinforcement element after deployment. Securing mechanisms may be self-expanding. Securing mechanisms may be formed from shape memory or superelastic materials (e.g., polymer, metal, or metal alloys). Securing mechanisms may be formed from swellable materials. Securing mechanisms may be formed from any combination of discussed or other suitable materials.

In some embodiments, securing mechanisms may include one or more tissue perforating elements. Tissue perforating elements may assist in inhibiting movement of a reinforcing element. At least one perforating element may be inserted or otherwise attached, partially or completely through at least one tissue wall. The perforating element may be coated with a material to positively effect hemostasis, healing, and/or for any other purpose.

Reinforcing element 908 may be preshaped from biocompatible materials that substantially inhibit deformation of the reinforcing element. In some embodiments, an externally placed reinforcing element may be formed from one solid continuous piece of biocompatible material. In some embodiments, a reinforcing element may be formed from more than one type of material.

In some embodiments, a reinforcing element may be fabricated using several methods and processes including sintering, molding (e.g., injection molding), casting, adhesive bonding, laminating, dip coating, spraying as well as composites and combinations thereof and combinations of other suitable methods and processes.

In some embodiments, a reinforcing element may be formed from woven or knitted Dacron polyester. In some embodiments, a reinforcing element may be formed from woven or knitted biocompatible fabric. In some embodiments, a reinforcing element may be formed from polymer, metal, metal alloy, or a combination or other suitable material. Reinforcing elements may be formed from autogenous/autologous, and/or synthetic biocompatible materials. Synthetic biocompatible materials may include silicone, rubber, polyurethane, polytetrafluoroethylene (PTFE), expanded polytetrafluoroethylene (ePTFE), polyester, Dacron™, Mylar™, polyethylene, PET (Polyethylene terephthalate), polyamide, polyamide, PVC, Kevlar™ (polyaramid), polyetheretherketone (PEEK), polypropylene, polyisoprene, polyolefin, or a composite of these or other suitable materials.

In some embodiments, a reinforcing element may be partially or completely made from many different types of biodegradable/bioerodible/bioabsorbable materials. Biodegradable/bioerodible/bioabsorbable materials may include modified starches, gelatins, cellulose, collagen, fibrin, fibrinogen, connective proteins or natural materials (e.g., elastin), polymers or copolymers (e.g., polylactide [poly-L-lactide (PLLA), poly-D-lactide (PDLA)], polyglycolide, polydioxanone, polycaprolactone, polygluconate, polylactic acid (PLA), polylactic acid-polyethylene oxide copolymers, poly(hydroxybutyrate), polyanhydride, polyphosphoester, poly(amino acids), poly(alpha-hydroxy acid)) or related copolymers of these materials, as well as composites and combinations thereof and combinations of other biodegradable/bioabsorbable materials.

A reinforcing element may be partially or completely fabricated from materials that swell or expand when they are exposed to a fluid (e.g., blood, another body fluid, or an infused fluid). These materials may include hydrophilic gels (hydrogels), foams, gelatins, regenerated cellulose, polyethylene vinyl acetate (PEVA), as well as composites and combinations thereof and combinations of other biocompatible swellable or expandable materials.

In some embodiments, a reinforcing element may include a surface coating. The surface coating may be formed from biocompatible materials. Applying a biocompatible surface coating to the reinforcing element may allow the reinforcing element to be formed from one or more potentially non-biocompatible materials. Non-biocompatible materials may be desirable for use due to other preferred properties (e.g., structural properties).

At least one coating may be located on a surface, as well as inside a reinforcement element. The reinforcement element may be coated with hydrophilic materials that are biologically inert and reduce surface friction (e.g., materials such as Parylene). One method to reduce surface tension for any metallic or metallic alloy elements or components of the reinforcement element is to chemically polish or electropolish those surfaces that will come in contact with blood or tissue. Sandblasting, beadblasting, or other known methods may be performed prior to polishing. Chemical polishing or electropolishing may reduce platelet adhesion because of the smooth surface that results. Chemical polishing and or electropolishing process may be used as an effective way to reduce the thickness of any metal or metal alloy device components.

The e.g., may incorporate one or more coatings, materials, compounds, substances, drugs, therapeutic agents, etc. that positively affect healing. Tissue adjacent the reinforcement element, at the tissue attachment site, at and or near where the reinforcement element is located may be positively affected by compounds incorporated into the reinforcement element. Compounds may be either incorporated into the structure forming the device, incorporated into a coating, contained within a reservoir on or inside the device, or a combination of locations. Thromoboresistance materials, antiproliferative materials, or other coatings intended to prevent thrombosis (acute and or chronic), hyperplasia, platelet aggregation, or other negative response, at or near the attachment of the reinforcement element may be incorporated and/or used in combination with the reinforcement element. The coatings, materials, compounds, substances, drugs, therapeutic agents, etc. may be used by themselves, and/or contained in a carrier such as a polymeric matrix, starch, or other suitable material or method. The coatings may be liquid, gel, film, uncured, partially cured, cured, combination or other suitable form.

Coatings on or in any component of the reinforcement element may be used to deliver therapeutic and pharmaceutic agents. Therapeutic and pharmaceutic agents may include (but are not limited to): antiproliferative/antimitotic agents including natural products such as vinca alkaloids (e.g., vinblastine, vincristine, and vinorelbine), paclitaxel, epidipodophyllotoxins (e.g., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (e.g., L-asparaginase which systemically metabolizes L-asparagine and deprives cells which do not have the capacity to synthesize their own asparagine); antiplatelet agents such as G(GP) II.sub.b/III.sub.a inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (e.g., cisplatin, carboplatin), procarbazine, hydroxyurea, mitotane, aminoglutethimide; hormones (e.g., estrogen); anticoagulants (e.g., heparin, synthetic heparin salts and other inhibitors of thrombin); fibrinolytic agents (e.g., tissue plasminogen activator, streptokinase and urokinase), aspirin, dipyridamole, ticlopidine, clopidogrel, abciximab; antimigratory; anti secretory (breveldin); anti-inflammatory: such as adrenocortical steroids (cortisol, cortisone, fluorocortisone, prednisone, prednisolone, 6.alpha.-methylprednisolone, triamcinolone, betamethasone, and dexamethasone), non-steroidal agents (salicylic acid derivatives i.e. aspirin; para-aminophenol derivatives (e.g., acetominophen; indole and indene acetic acids (e.g., indomethacin, sulindac, and etodalac)), heteroaryl acetic acids (e.g., tolmetin, diclofenac, and ketorolac), arylpropionic acids (e.g., ibuprofen and derivatives), anthranilic acids (e.g., mefenamic acid, and meclofenamic acid), enolic acids (e.g., piroxicam, tenoxicam, phenylbutazone, and oxyphenthatrazone), nabumetone, gold compounds (e.g., auranofin, aurothioglucose, gold sodium thiomalate); immunosuppressives: (e.g., cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor signal transduction kinase inhibitors. A clot promoter may be used, such as protamine sulphate or calcium hydroxide. Endothelial cells may also be added to the reinforcement element.

Therapeutic compounds/solutions may be blended with a reinforcement element's base materials during fabrication, applied just prior to deployment, and/or after the reinforcement element has been deployed. The therapeutic materials may be located on, through, and/or inside of the reinforcement element. Therapeutic materials may be located in holes, grooves, slots (or other indentations) and/or other designs. For example, the tissue contacting surface may have partial or complete holes, grooves, or other indentations, filled with a therapeutic substance, in contact with the host vessel tissue.

In some embodiments, a reinforcement element may be formed from multiple layers. The reinforcement element may include a reservoir. The reservoir may include therapeutic agents. Therapeutic agents may elute from the reservoir into tissue contacting a surface of the reinforcement element. In some embodiments, the reservoir may be connected to a tissue-perforating element or anchor.

A reinforcement element may be formed from any suitable material to stabilize, strengthen, cross link, arrest or delay the heart tissue remodeling process, or otherwise reinforce the infarcted myocardial tissue itself. A reinforcement element may include binding agents, collagen (e.g., through direct infusion, and or as a result of tissue heating), therapeutic substance, pharmacologic compounds or materials, radioactive material, genetic material (e.g., stem cells, etc.), a combination of any of the above, or any other suitable material(s).

Securing mechanisms such as coils, or other anchors may also have partial or complete holes, slots, grooves, or other openings filled with a therapeutic substance, or simply coated on the outside surfaces. Designs such as this may allow tissue direct contact with therapeutic substances, while maintaining the functional ability of the device or securing mechanism. Combinations of therapeutic substances or coatings may be used on the same device. For example, a more viscous (gel or other) therapeutic substance may be used to fill the partial or complete holes (or other) on the securing mechanisms, while the main body of the devise is coated with a less viscous (liquid) material. Therapeutic substances may be the same, or a combination of more than one type used on the device and/or securing mechanisms. The coatings may be designed to provide benefits acutely, and/or over a period of time. The coatings, materials, compounds, substances, therapeutic agents, etc. may be desired to be static, and/or eluting. The coatings, materials, compounds, substances, therapeutic agents, etc. may elute from the coated (or embedded) device (or component) over time and enter the surrounding and or adjacent tissue. The coatings, materials, compounds, substances, drugs, therapeutic agents, etc. may remain on the reinforcement element for at least three days, and up to approximately six months or longer.

Post device fabrication coating methods may include, but are not limited to, spin coating, RF-plasma polymerization, dipping, spraying, brushing, submerging the device into a beaker containing a therapeutic solution while inside a vacuum chamber to permeate the device material, etc.

A reinforcement element may be partially or completely fabricated from materials that swell or expand when they are exposed to a fluid (e.g., blood, another body fluid, or an infused fluid). These materials may include hydrophilic gels (hydrogels), foams, gelatins, regenerated cellulose, polyethylene vinyl acetate (PEVA), as well as composites and combinations thereof and combinations of other biocompatible swellable or expandable materials.

In some embodiments, a reinforcement element may be permanently or temporarily attached to the inside and/or outside of the heart (Percutaneous Transluminal and Surgical system versions). In some embodiments, a reinforcement element may be deployed using a delivery/deployment device (e.g., endoscope, catheter, or other suitable device). A delivery/deployment device may be advanced to the surface of the ventricle (inside and or outside of the heart).

In some embodiments, a reinforcement element may be employed using implanted marker elements (or other) as a guide. The reinforcement element may be deployed and positioned along the margins of the akinetic/dyskinetic tissue. The margins of the akinetic/dyskinetic tissue may be represented by the marker implants (using fluoroscopy or other suitable visualization method) and or other suitable method.

In some embodiments, a reinforcement element may be attached to a wall of a ventricle. Once a physician is satisfied with the placement of the reinforcement element, the delivery/deployment device may be withdrawn from the body and the access closed.

In some embodiments, a reinforcement element may be permanently or temporarily attached to the heart. For example, the reinforcement element may be removed after the active treatment/stabilization therapy has been completed. Heat and or therapeutic infusion may be made in the area of, and surrounding the, infarcted myocardial tissue, before, during and/or after the attachment of the reinforcement element. Heat methods may include a probe, catheter, or other suitable instrument with the capability to heat selected tissue. Methods of heating may include radio frequency (RF), ultrasound, microwave, laser, heat, localized delivery of chemical or biological agents and light-activated agents, combination or other suitable methods.

In some embodiments, a system for marking and evaluating at least a portion of a heart may include reference markers. Reference markers may be positioned using a deployment device. A deployment device may include a catheter-based version and/or surgical versions.

Reference markers may be positioned in heart tissue. Reference markers may be used to identify the margins between healthy and akinetic or dyskinetic heart tissues. Reference markers may be used to track disease progression. Reference markers may be used as a guide for plication, patch placement, or other reinforcement element location and attachment. Reference markers may be used as a guide to reinforce, support or exclude the akinetic/dyskinetic heart tissue from the healthy tissue.

The reference markers themselves may be in the form of at least one wire, rod, bar, a combination, or other suitable geometry and design. The reference markers may be formed from any material that is able to be visualized using fluoroscopy, ultrasound, MRI, or any other suitable visualization means.

A system may include a deployment device. The deployment device may include a catheter (or surgical instrument) capable of inserting reference markers along the margin between healthy and akinetic/dyskinetic heart tissue. The catheter may have a lumen with at least one preloaded reference marker. The reference marker may be deployed past the distal end of the deployment device, and into the heart tissue. The reference marker may be deployed by way of a proximal thumb-slide attached to a stylet that engages and advanced the proximal end of a reference marker.

The catheter or surgical instrument may include a fixed and or removable device which functions to evaluate adjacent tissue and to assist with the determination as to where the marking implant should be deployed and inserted into the margin tissue.

Akinetic/dyskinetic tissue location techniques may include using anatomical structures (e.g., as a guide), visualization/sensors, reference markers, or a combination of any of these methods. Systems which may be used to accomplish these various methods may include visualization, CARTO XP System (Biosense Webster—3D Ultrasound), fiber optic, fluoroscopic markers, intravascular ultrasound (2D IVUS), or MRI.

Methods may be employed which assist in confirming tissue contact between, for example, tissue and a deployment device and/or reference markers. Tissue contact may be confirmed using, for example, doppler sensor, electrical impedance, and/or ultrasonic transducer.

As noted above, one representative version of the tissue irradiating reinforcement element may apply ionizing radiation to the surface of the infarcted tissue. Optimally, the radiation has a range which extends through the ventricular wall but not significantly beyond. An example of a radioactive source that can apply this type of radiation dosing is a beta particle emitting radioisotope such as phosphorous-32 which has a range of approximately 3.5 mm for 90% of the electrons that it emits. Phosphorous-32 has a half-life of 14.3 days which means that it has a very high rate of specific activity. Even very small amounts of phosphorous-32 can provide a sufficiently high level of irradiation to the myocardial tissue. The radioactive patch would typically be an elongated flexible structure which can be applied in the region and covering the infarcted myocardial tissue. Typically, the radioactive bandage would extend for approximately 1 to 5 mm beyond the infarct area in all directions.

The radioactive patch may include a shield structure which surrounds the thin, elongated radioactive portion thus disallowing stray radiation to any surrounding or adjacent tissue. The purpose of the shield may be to absorb beta particles without the creation of a significant level of bremsstrahlung. Virtually any elastomer may serve that purpose. The shield may be a high density source which is placed to substantially reduce any stray radiation. The shield may typically be formed from a high density metal such as tungsten impregnated into any one of several elastomers. The purpose of the shield would be to absorb any photon emission caused by bremsstrahlung which resulted from a beta particle hitting the nucleus of some atom. If the radioactive source was a low energy x-ray emitter, the radiation shields might be combined into a single shield having a high density metal impregnated into some elastomer. A radiation dose applied to the inside, and or outside of the ventricular wall of between 500 and 3,000 cGy may be sufficient for most applications.

The heart wall reinforcing devices and accessories described herein (with or without modifications) can be used during cardiopulmonary supported, beating heart, stopped heart, open field, closed-chest, minimally invasive, port, endoscopic, laparoscopic and or robotically assisted surgery, any combination thereof, or other cardiovascular technique. The devices detailed in this system overview may be a hand-held device used through a median sternotomy, lateral thoracotomy, intercostals, port-access, mini-sternotomies, other less invasive approaches involving subxiphoid access, inguinal approaches, or sub-thoracic approaches adjacent the diaphragm. Alternatively, the systems described herein may be modified for catheter based applications by elongating the patch deployment shaft, altering the dimensions of the device, and incorporating other features tailored for intravascular access and internal heart deployment and tissue attachment.

In an operation, a surgeon determines what size, shape, and orientation he intends to reconstruct a ventricle (the “intended” or appropriate shape). During a surgical procedure, the surgeon then opens the ventricle and notes the extent of the scar inside the ventricle. Reinforcing element 908 may be placed in the ventricle. A surgeon may mark the extent of the scar tissue on reinforcing element 908. Reinforcing element 908 may be removed. Upon removal, excess material may be trimmed from reinforcing element 908. Reinforcing element 908 is placed back in the ventricle and the surgeon ensures that the apex of the patch is located at the apex of the ventricle. Reinforcing element 908 may be sutured into the ventricle, excluding much, if not all, of the akinetic tissue and creating a new apex. In some embodiments, it may be possible and/or desirable to exclude all of the akinetic tissue.

In an embodiment of the procedure, a surgeon may decide on the volume of the ventricle. The surgeon may use a shaping device and a matching reinforcing element. After the ventricle is open, a Fontan stitch may be placed on the border zone of the akinetic and viable tissue. The shaping device may be introduced into the ventricle and the Fontan stitch is pulled tight. A portion of the shaping device may project out of the Fontan stitch. Reinforcing element 908 may be cut such that the shape of the patch matches the projection of the shaping device outside of the Fontan stitch. Cut reinforcing element 908 may be sewn to a portion of the heart hemostatically. The shaping device may be removed prior to completely sewing reinforcing element 908. Such a procedure would yield a reconstructed ventricle of the size and shape that is substantially the same as the intended or appropriate shape. FIG. 26 depicts an embodiment of a sectional view of dilated heart with a reinforcing element (e.g., an apical patch).

In certain embodiments, a reinforcing element may be delivered to the heart in a percutaneous method. The method may include a catheter. The catheter may be guided through a vascular structure of a human body to a portion of the heart (e.g., the left ventricle). In some embodiments, a catheter may be guided with the assistance of fluoroscopy and/or echocardiography. Upon reaching a portion of the heart (e.g., the left ventricle), the catheter is positioned approximate to a predetermined attachment point (e.g., the apex of the left ventricle in the case of an apical infarction). A guidewire may be pushed out of the catheter, and the guidewire is attached to the point of interest.

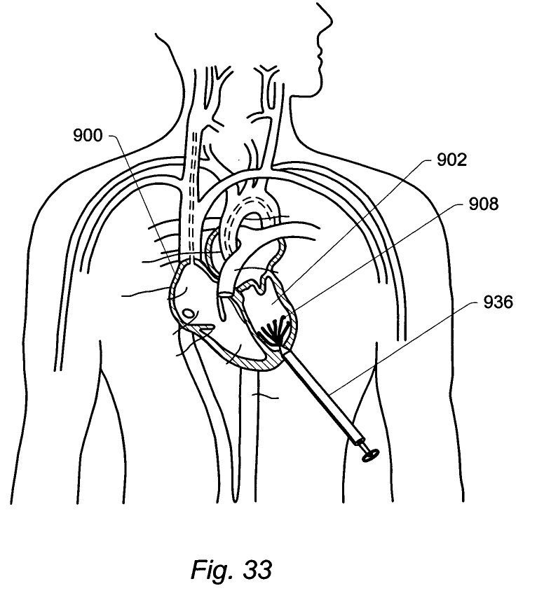

In certain embodiments, a reinforcing element may be delivered to a heart by a surgical procedure including, but not limited to, a thoracotomy. FIG. 33 depicts a representation of an embodiment of a method for positioning reinforcing element 908 using a thoracotomy. Reinforcing element 908 may be delivered through cannula 936 positioned in an incision in the chest. The cannula may be inserted through, for example, an apex of left ventricle 902 of heart 900 in order to position reinforcing element 908 on an endocardial surface of the left ventricle.

A reinforcing element may be deployed through the distal end of the catheter once the catheter is in position. The reinforcing element may be in a retracted or inactivated state. In some embodiments, a reinforcing element may include substantially flexible materials (e.g., shape memory materials) allowing the reinforcing element to be change shape (e.g., collapsed) so that the reinforcing element may more easily pass through a constricted opening (e.g., a catheter). Upon deployment, a positioning enabler (e.g., a wire) may be employed to assist in properly positioning the reinforcing element. Once positioned, a user (e.g., surgeon) may employ means known to one skilled in the art to assess the position of reinforcing element. For example, the reinforcing element may include markers (e.g., radioscopic). In some embodiments, alignment of the reinforcing element to the portion may be performed by direct visualization of the portion of the heart (typically including scar and/or non viable tissue) and the reinforcing element with a fiber optic catheter. The fiber optic catheter may emit electromagnetic waves of certain frequencies that are transparent to the blood pool. The reflection may be captured by a different fiber optic line (possibly in the same catheter) and presented on the monitor.

After deployment, the reinforcing element may be turned to a proper orientation. Note that from earlier cardiac analysis prior to the intervention, the location of the scar with respect to other cardiac structures is known. So based on relative location of the scar with respect to other cardiac landmarks, the device may be aligned with the scar, for example, under fluoroscopic or echocardiographic guidance.

The user may activate the reinforcing element to attach the reinforcing element to the adjacent tissue before assessing the position of the reinforcing element. In some embodiments, the user may assess the position of the reinforcing element before activating the reinforcing element. Upon assessment of the position of the reinforcing element, the user may decide to reposition the reinforcing element to provide better structural support. The reinforcing element may be releasably attached to the portion of the heart to allow repositioning of the reinforcing element.

A method for delivering a reinforcing element to a portion of the heart may include at least one guidewire. The guidewire may be delivered to the heart in a percutaneous method. A distal end of the guide wire may be positioned in the heart. In some embodiments, a guidewire may be positioned before a catheter. The catheter may be inserted through the vasculature over the guidewire using the guidewire to assist in properly positioning a distal end of the catheter in the heart. In some embodiments, a distal end of a catheter may be positioned in a portion of a heart before a guidewire. A guidewire may already be positioned in the catheter before the catheter is positioned and/or the guidewire may be inserted into the heart through the catheter after the catheter is positioned.

In some embodiments, a reinforcing element may be positioned using a guidewire to assist in placement of the reinforcing element. The reinforcing element (which is in retracted position) may be pushed out from a catheter over the guidewire into the ventricle. The reinforcing element may be turned to properly orient it with the scar and/or non viable tissue.

In some embodiments, a guidewire may include a coupling mechanism positioned towards a distal end of the guidewire. Coupling mechanisms may include a clip for example. The coupling mechanism may be remotely operated. Upon positioning the distal end of the guidewire substantially adjacent a target area of a heart a user wishes to reinforce, the coupling mechanism may be activated. The coupling mechanism may function to at least couple the guidewire to the target area. Coupling the coupling mechanism to the target area may assist in positioning a reinforcing element.

Upon satisfactory implantation of the reinforcing element, the guidewire and the catheter may be removed from the ventricle.

In some embodiments, reinforcing elements described herein may be used in combination with other methods. Other methods may include, for example, removing autologous muscle cells, stem cells, etc., and culturing the cells to generate implantation cells necessary for myocardial repair. Cultured cells may be implanted via injection or the like into the myocardium. In the myocardium, cultured cells may have an opportunity to generate new heart muscle. One of the factors that renders ischemic heart disease so devastating is the inability of the cardiac muscle cells to divide and repopulate areas of ischemic heart damage. As a result, cardiac cell loss as a result of injury or disease is irreversible. Implanted cells may overcome the inability of cardiac muscle cells to divide by thriving in an oxygen deprived infarct area. Methods for cell implantation, as well as associated apparatus, are available commercially from such companies as Bioheart Inc. in Weston, Fla. (www.bioheartinc.com).

A reinforcing element may be used in combination with autologous cells. A reinforcing element may be used to reinforce a portion of an endocardial surface of a heart. Structurally reinforcing the portion may allow other tissue rehabilitation techniques to function more effectively. For example, structural reinforcement of the portion may alleviate stress on the portion of the tissue while the injured tissue is treated by one or more of a variety of methods. In some embodiments, the tissue may be treated with autologous cells to generate new heart muscle tissue. Structural reinforcement (e.g., with a reinforcing element) may facilitate faster generation of new heart muscle tissue. In some embodiments, hormones and/or medicants may be used to facilitate healing and/or regeneration of heart muscle tissue.

In some embodiments, a reinforcing element may be attached to a portion of an external surface of a heart. A reinforcing element may be used to inhibit further deformation of at least a portion of a human heart. The reinforcing element may be positioned over infarcted tissue, coupling the reinforcing element to an external surface of the heart. Coupling the reinforcement element to an external surface of the heart such that a majority or all of the infarcted tissue is covered may inhibit further deformation of the heart. In some embodiments, the reinforcement element may be designed such that it covers all of the infarcted tissue as well as an area surrounding the infarcted tissue. The reinforcement element may extend, for example, 1 to 5 mm beyond the area of the infarcted tissue.

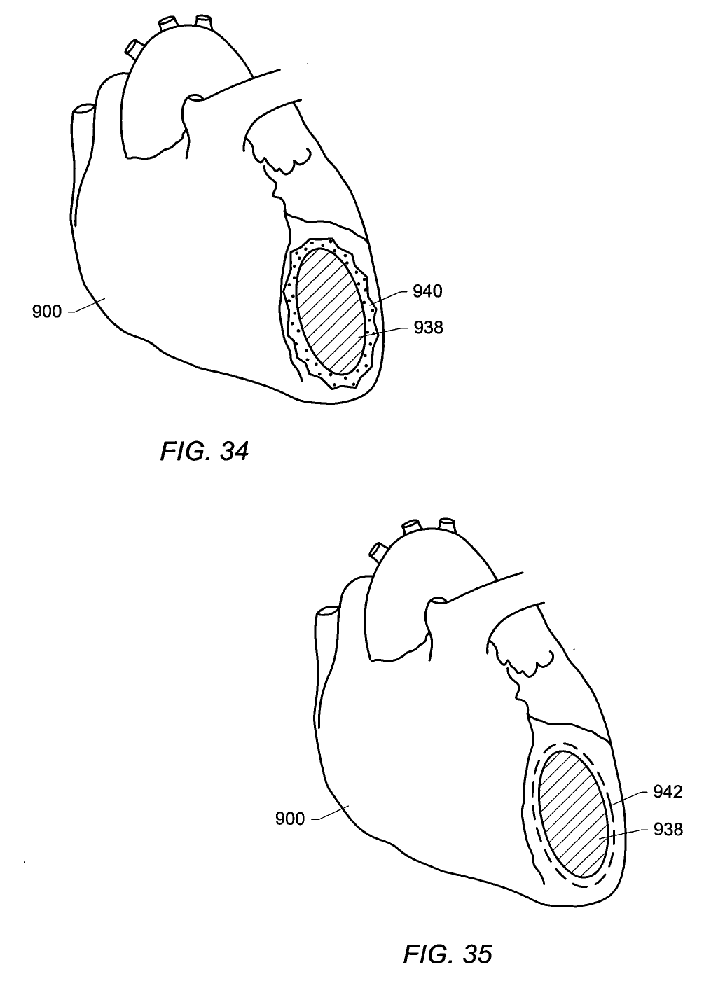

FIG. 34 depicts an embodiment of a human heart 900 including a depiction of infarcted tissue 938 in the shaded area of the heart with a border zone of tissue 940 surrounding the infarcted area of the heart. FIG. 35 depicts an embodiment of a human heart 900 with an area of infarcted tissue 938 as well as a broken line 942 indicating where a reinforcement element may be positioned and/or coupled to.

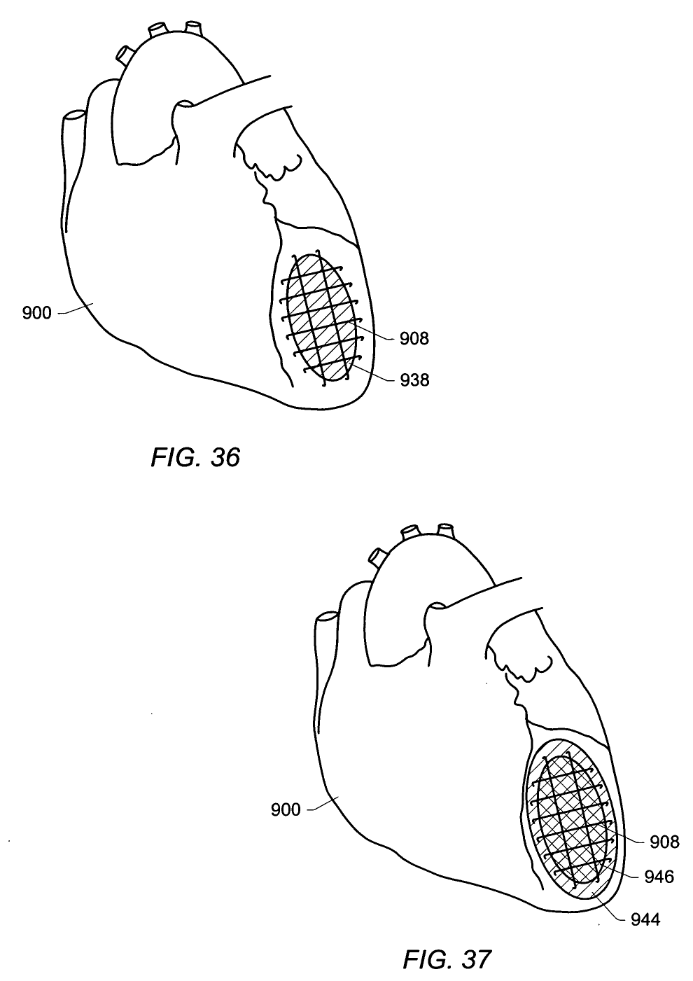

In some embodiments, upon positioning and coupling a reinforcement element to a portion of a heart, the reinforcement element may be employed to at least partially reshape a portion of the heart. FIG. 36 depicts an embodiment of a human heart 900 with suture or staples (embodiments of a reinforcement element 908) coupled to the heart over infarcted tissue 938. The reinforcement element may be employed to adjust the shape and/or volume of a portion of the heart. For example, the reinforcement element may be composed of sutures, the sutures may be tightened such that the infarcted tissue is drawn together as well as effectively reshaping the heart. FIG. 37 depicts an embodiment of a human heart 900 with suture or staples (embodiments of a reinforcement element 908) coupled to the heart over the infarcted tissue, before and after the infarcted area has been reduced. The outer portion of the shaded area represents the infarcted portion before adjustment 944 using the reinforcement element. The inner portion of the shaded area represents the infarcted portion after adjustment 946 using the reinforcement element (e.g., after the reinforcement element has been activated), “crimping” the infarcted tissue and reshaping the heart.

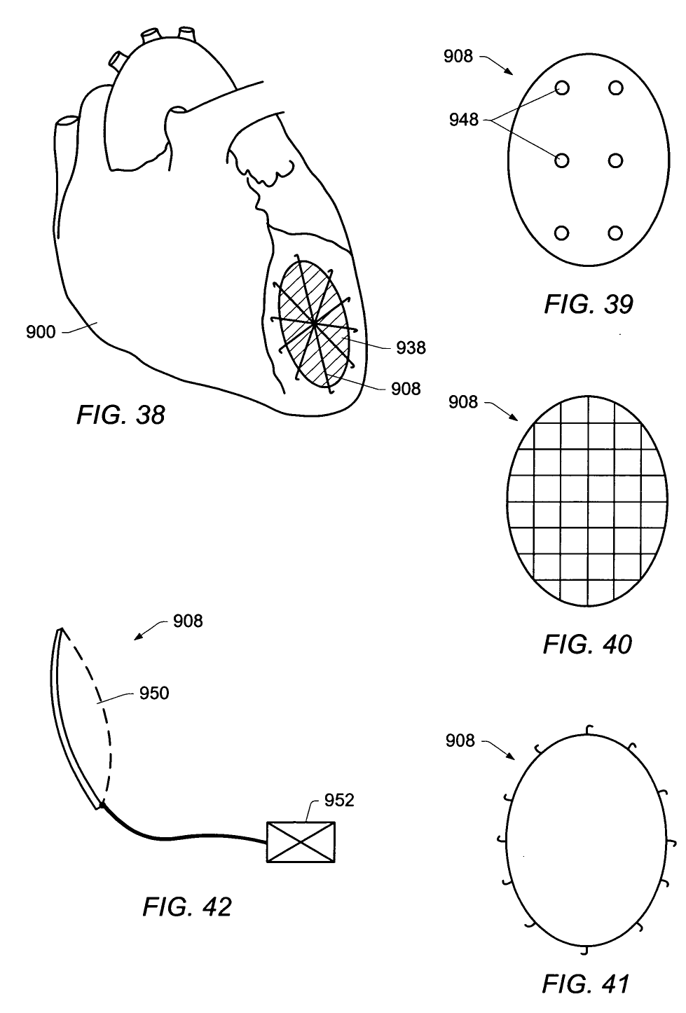

FIG. 38 depicts an embodiment of a human heart 900 with suture or staples (embodiments of a reinforcement element 908) coupled to the heart over the infarcted tissue 938 before the infarcted area has been reduced. FIG. 38 depicts an embodiment a reinforcement element using elongated members (e.g., sutures, staples) coupled together in a center of the reinforcement element. In this embodiment, radial forces may be used upon activation of the reinforcement element to reshape the heart.

FIGS. 39-42 depict different embodiments of reinforcement elements 908. FIG. 39 depicts an embodiment of a patch 908 including openings 948. Elongated members (e.g., sutures, staples) may be positioned (e.g., looped) through the openings in the reinforcement element and subsequently activated such that the heart is reshaped. FIG. 40 depicts an embodiment of a wire mesh patch 908. The wire mesh patch may include mechanisms to activate the patch such that the patch exerts pressure or force on the tissue it is coupled to such that the heart is reshaped. FIG. 41 depicts an embodiment of a patch 908 including coupling mechanisms. The coupling mechanisms may include barbs, hooks, and/or anchors.

In some embodiments, mechanisms used in combination with a reinforcement element may be used to assist in reshaping at least a portion of a heart. FIG. 42 depicts an embodiment of a patch 908 including an inflatable/expandable portion 950. In some embodiments a reinforcement element may include an inflatable portion. The inflatable portion may be positioned between the outer surface of the patch and the infarcted heart tissue. The reinforcement element may include an inflation mechanism 952 which functions to inflate the inflatable portion upon activation.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An apparatus for reshaping at least a portion of a human heart, comprising:
a reinforcing element configured to attach to a portion of the epicardial surface of a left or right ventricle of the heart, wherein the reinforcing element is configured to change a size and/or shape of an interior space in a left or right ventricle.

2. (canceled)

3. The apparatus of claim 1, wherein the reinforcing element comprises one or more portions configured to facilitate post operative evaluation of a positioned reinforcing element.

4. (canceled)

5. (canceled)

6. The apparatus of claim 1, wherein the reinforcing element is attached to a portion of the epicardial surface using sutures.

7. The apparatus of claim 1, wherein the reinforcing element is attached to a portion of the epicardial surface using staples.

8. (canceled)

9. The apparatus of claim 1, wherein the reinforcing element is configured to inhibit expansion of an average of an endocardial surface over a cardiac cycle of the left or right ventricle.

10. The apparatus of claim 1, wherein the reinforcing element is configured to inhibit expansion of an endocardial surface such that normal contraction and expansion during a cardiac cycle of the heart remains substantially unimpeded.

11. The apparatus of claim 1, wherein the reinforcing element is configured to releasably attach to a portion of the epicardial surface of the heart.

12. The apparatus of claim 1, further comprising an adjustment mechanism, wherein the adjustment mechanism is configured, upon activation, to change a dimension of at least a portion of the reinforcing element.

13. The apparatus of claim 1, further comprising an adjustment mechanism, wherein the adjustment mechanism comprises an inflatable portion configured, upon activation, to change a dimension of at least a portion of the reinforcing element.

14. (canceled)

15. (canceled)

16. The apparatus of claim 1, further comprising an adjustment mechanism, wherein the adjustment mechanism is configured, upon activation, to change a dimension of at least a portion of the reinforcing element, and further comprising an engagement mechanism configured to inhibit the activated adjustment mechanism from moving.

17. The apparatus of claim 1, further comprising an activation mechanism, wherein the activation mechanism is configured to attach the reinforcing element to a portion of the surface of the heart.

18. The apparatus of claim 1, wherein the reinforcing element comprises a patch.

19. (canceled)

20. The apparatus of claim 1, wherein the reinforcing element comprises a patch, wherein the patch comprises two or more openings, wherein the openings are configured to allow elongated members to pass through the openings, and wherein upon activation the elongated members are configured to change a dimension of at least a portion of the reinforcing element, and to thereby change a dimension of at least a portion of the ventricle.

21. (canceled)

22. (canceled)

23. (canceled)

24. The apparatus of claim 1, wherein the reinforcing element comprises a patch, wherein the patch comprises a system of elongated members, and wherein upon activation the elongated members are configured to change a dimension of at least a portion of the reinforcing element, and to thereby change a dimension of at least a portion of the ventricle.

25. (canceled)

26. (canceled)

27. The apparatus of claim 1, wherein the reinforcing element comprises shape memory materials.

28. (canceled)

29. The apparatus of claim 1, wherein the portion of the epicardial surface comprises at least some scar tissue.

30. The apparatus of claim 1, wherein the portion of the epicardial surface comprises at least some infarcted tissue.

31-90. (canceled)

91. A method for reshaping at least a portion of a human heart, comprising:
attaching a reinforcing element to a portion of the epicardial surface of a left or right ventricle of the heart such that
a size and/or shape of an interior space in a left or right ventricle is changed.

92-113. (canceled)

114. The apparatus of claim 1, wherein the reinforcing element is configured to change from a first shape to a second shape upon activation changing a size and/or shape of an interior space in a left or right ventricle.

115. The method of claim 91, further comprising activating the reinforcing element such that the reinforcing element changes from a first shape to a second shape.
